(19) United States
(12) Patent Application Publication
(10) Pub. No.: US 2026/0226472 A1
(43) Pub. Date: Aug. 6, 2026

(54) COMPOSITIONS FOR MODULATING KRAS EXPRESSION AND USES THEREOF

(71) Applicant: MOLECULAR AXIOM, LLC, San Diego (CN)

(72) Inventors: Lishan CHEN, San Diego, CA (US); Yuching CHEN, San Diego, CA (US); Rosaline Do CARSON, San Diego, CA (US); Bohan JIN, San Diego, CA (US)

(21) Appl. No.: 19/149,303

(22) PCT Filed: Jan. 18, 2024
(86) PCT No.: PCT/US2024/011938

§ 371 Date: Jul. 18, 2025

Related U.S. Application Data
(60) Provisional application No. 63/480,467, filed on Jan. 18, 2023.

(51) Int. Cl. C12N 15/113; A61P 35/00

(52) U.S. Cl. CPC C12N 15/1137; A61P 35/00; C12Y 306/05002; C12N 2310/11; C12N 2310/315; C12N 2310/321; C12N 2310/322; C12N 2310/3231; C12N 2310/341

(57) ABSTRACT

Described herein are compounds, compositions, and methods for modulating KRAS expression, such as mutated KRAS expression, and/or downstream signaling pathways. Also described herein are compounds, compositions, and methods for treating a disease or condition associated with mutated KRAS. In some embodiments, the compound comprises at least one antisense oligonucleotide that, upon being delivered into a cell, hybridizes to an endogenous KRAS mRNA, which leads to the degradation of the KRAS mRNA. In some embodiments, the antisense oligonucleotide hybridizes to an mRNA encoding a mutated KRAS protein, such as a KRAS protein comprising a G12C mutation, a G12V mutation, a G12D mutation, or a G12A mutation.

### PRIORITY

This application claims the benefit of, and priority to, U.S. Provisional Application No. 63/480,467, filed Jan. 18, 2023, the contents of which are hereby incorporated by reference in its entirety.

### SEQUENCE LISTING

The contents of the text file submitted electronically herewith are incorporated herein by reference in their entirety: A computer readable format copy of the Sequence Listing (filename: “MAX-008PC_133279-5008_SequenceListing”; date recorded: Jan. 17, 2024; file size: 45,380 bytes).

### BACKGROUND

Certain diseases or conditions are caused by genetic mutations or deregulated signaling pathways. To treat such diseases or conditions, one of the most sought after treatment options involves direct editing of the genetic mutations, or transcriptional/translational regulation using gene silencing tools or methods. Oligonucleotide-induced gene silencing can control RNA expression of target genes in various embodiments including transcription inactivation, mRNA degradation, transcriptional attenuation. Existing RNA-induced gene silencing tools can be inefficient and/or not specific. Therefore, there remains a need for compositions and methods for effective control of gene expression, and particularly to reduce RNA levels of mutated genes such as KRAS and/or downstream signaling pathways.

### SUMMARY

In aspects and embodiments, there is provided a compound comprising an antisense oligonucleotide that inhibits the expression of a KRAS mRNA, wherein the antisense oligonucleotide comprises 10 to 30 linked nucleotides and has a sequence that is complementary to a KRAS mRNA, and wherein the oligonucleotide has at least 8 contiguous nucleotides of any one of SEQ ID NOS: 1-10 and 14-21.

In embodiments, the oligonucleotide is at least 12 nucleotides in length. In embodiments, the oligonucleotide is at least 14 nucleotides in length. In embodiments, the oligonucleotide is from 10 to 24 nucleotides in length, or 10 to 16 nucleotides in length, or 12 to 16 nucleotides in length.

In embodiments, the oligonucleotide is 12, 13, 14, 15, or 16 nucleotides in length. In embodiments, the oligonucleotide is 14 nucleotides in length. In embodiments, the oligonucleotide comprises at least 12 contiguous nucleobases of any one of SEQ ID NOs: 1-10 and 14-21. In embodiments, the oligonucleotide comprises or consists of a nucleobase sequence of any one of SEQ ID NOs: 1-10 and 14-21. In embodiments, the oligonucleotide has a nucleobase sequence selected from SEQ ID NOs: 2, 3, 4, 5, 6, 7, 8, 9, and 10.

In embodiments, the antisense oligonucleotide has a stretch of at least 6 DNA nucleotides sufficient to recruit RNaseH. In embodiments, one or more DNA nucleotides comprise a 2′ chemical modification independently selected from 2′-Fluoro, 2′-Methyl, and 2′-Ethyl. In embodiments, DNA nucleotides do not comprise a 2′ chemical modification. In embodiments, the antisense oligonucleotide is a gapmer having a 5′ and a 3′ segment, each of the 5′ and 3′ segments being from 2 to 6 nucleotides or from 2 to 4 nucleotides, and where the 5′ and 3′ segments do not contain DNA nucleotides. In embodiments, the 5′ and 3′ segments are each independently selected from 2 or 3 nucleotides in length, and the 5′ and 3′ segments flank an internal sequence of 8 DNA nucleotides. In embodiments, one or more nucleotides of the 5′ segment and the 3′ segment comprise 2′-O substituents, optionally where all of the nucleotides of the 5′ segment and the 3′ segment comprise 2′-O substituents. In embodiments, the 2′-O substituents are independently selected from 2′-O methyl, 2′-O ethyl, 2′-O methoxyethyl (MOE), and a bridged nucleotide having a 2′ to 4′ bridge. In embodiments, the bridged nucleotide has a methylene bridge (LNA) or a constrained ethyl bridge (cEt). In embodiments, the antisense oligonucleotide has a modified backbone. In embodiments, the antisense oligonucleotide comprises one or more phosphorothioate or phosphorodithioate nucleotides. In embodiments, the oligonucleotide is fully phosphorothioate or phosphorodithioate linked. In embodiments, the oligonucleotide is fully phosphorothioate linked.

In embodiments, cytosine nucleobases in the antisense oligonucleotide are modified cytosine, which is optionally 5-methyl cytosine or 5-hydroxymethyl cytosine. In embodiments, the antisense oligonucleotide has a nucleobase sequence and structure (i.e., modification pattern) shown in one or more of Tables 1, 2, 3, 6, 7, 8, 9, 10, and 11. In embodiments, the compound further comprises a cell targeting or penetrating moiety. In embodiments, the cell targeting or penetrating moiety is conjugated directly or indirectly at the 3′ end of the oligonucleotide, optionally though a linker. In embodiments, the moiety comprises a sterol conjugate or fatty acid conjugate, which is optionally cholesteryl, palmitoyl, or stearyl conjugate. In embodiments, the compound further comprises a cell targeting aptamer.

In embodiments, the compound does not comprise any encapsulation or transfection reagent. In embodiments, the antisense oligonucleotide is encapsulated in a particle. In embodiments, the particle is a liposome, polymeric nanoparticle, or lipid nanoparticle. In embodiments, the compound is formulated for parenteral administration. In embodiments, there is provided a pharmaceutical composition comprising a compound of the present disclosure and a pharmaceutically acceptable carrier or vehicle.

In embodiments, there is provided a method for treating a subject having a condition associated with aberrant expression of KRAS or associated with mutated KRAS, comprising administering an effective amount of the compound of the present disclosure or a pharmaceutical composition of the present disclosure to the subject. In embodiments, the subject has a malignancy associated with an abnormality of KRAS-mediated signaling pathway. In embodiments, the malignancy is associated with KRAS or a mutated KRAS. In embodiments, the mutated KRAS mRNA encodes a mutated KRAS protein comprising a G12C mutation. In embodiments, the mutated KRAS mRNA encodes a mutated KRAS protein comprising a G12D mutation or a G12V mutation. In embodiments, the malignancy associated with an abnormality of KRAS-mediated signaling pathway is non-metastatic. In embodiments, the malignancy associated with an abnormality of KRAS-mediated signaling pathway is metastatic.

In embodiments, the malignancy is a carcinoma. In embodiments, the malignancy is breast cancer, cervical cancer, pancreatic cancer, squamous cell carcinoma, head and neck cancer, thyroid cancer, gastric cancer, colon cancer, or liver cancer. In embodiments, the malignancy is pancreatic cancer. In embodiments, the malignancy is cervical squamous cell carcinoma, endocervical adenocarcinoma, cholangiocarcinoma, esophageal carcinoma, colon adenocarcinoma, oral squamous cell carcinoma, head and neck squamous cell carcinoma, lung adenocarcinoma, lung squamous cell carcinoma, non-small cell lung cancer, liver hepatocellular carcinoma, lung squamous cell carcinoma, rectum adenocarcinoma, stomach adenocarcinoma, thyroid carcinoma, or pancreatic adenocarcinoma. In embodiments, the malignancy is pancreatic cancer, such as pancreatic adenocarcinoma. In embodiments, the antisense oligonucleotide is administered parenterally.

### BRIEF DESCRIPTION OF THE DRAWINGS

FIG. 1 illustrates knockdown of KRAS mRNA encoding a mutated KRAS protein comprising the G12C mutation mediated by an antisense oligonucleotide (ASO) described herein, from left to right STN-016 (a), STN-017 (b), STN-018 (c), STN-019 (d), STN-001 (e), STN-002 (f), and STN-003 (g).

FIG. 2 illustrates three-dimensional (3D) cell proliferation inhibition due to inhibiting expression of mutated KRAS by contacting the cell harboring the KRAS G12C mutation (Mia Paca-2) with an oligonucleotide described herein, from left to right STN-022 (a), STN-016 (b), STN-017 (c), STN-025 (d), STN-018 (e), STN-020 (f), STN-021 (g), STN-019 (h), STN-001 (i), STN-002 (j), and STN-003 (k).

FIG. 3 illustrates a western blot analysis after G12V KRAS knockdown with ASOs in LCLC-97TM1 Cell Line. For reference, the ASOs loaded from left to right are STN-100080, STN-100993, STN-100994, STN-100987, and STN-100989.

### DETAILED DESCRIPTION

Described herein are compounds, compositions, and methods for modulating KRAS expression (e.g., mutated KRAS expression) and/or downstream signaling pathways. Also described herein are compounds, compositions, and methods for treating a disease or condition by modulating the gene expression or signaling pathways associated with the disease or condition. In some embodiments, the compound comprises at least one antisense oligonucleotide that, upon being delivered into a cell, binds (e.g., hybridizes) to an endogenous KRAS mRNA, which leads to the degradation of the KRAS mRNA. In some embodiments, described herein is a method for utilizing the compound or the oligonucleotide described herein. In some embodiments, the methods are used to treat the disease or condition by contacting a cell with the oligonucleotide to decrease KRAS gene expression (e.g., mutated KRAS) or an associated signaling pathway. In some embodiments, the antisense oligonucleotide hybridizes to an mRNA encoding a mutated KRAS protein, such as a KRAS protein comprising a G12C mutation, a G12V mutation, a G12D mutation, or a G12A mutation.

In some embodiments, the oligonucleotide is an antisense oligonucleotide, where the oligonucleotide is complementary and binds (e.g., hybridizes) to a segment of at least one endogenous nucleic acid (e.g., an mRNA). In some embodiments, the binding of the oligonucleotide to the endogenous nucleic acid leads to degradation of the endogenous nucleic acid or blocking of translation of the target protein from the endogenous nucleic acid, hence decrease of the expression of the gene encoded by the endogenous nucleic acid. For example, the binding of the oligonucleotide to the endogenous mRNA creates a duplex nucleic acid molecule, which can then recruit endogenous nuclease (e.g., RNaseH) for degradation of the mRNA.

In some embodiments, the oligonucleotide comprises a stretch of DNA nucleotides (such as a stretch of 5 to 10 DNA nucleotides) sufficient to recruit RNaseH when hybridized to the target mRNA. In embodiments, the stretch of DNA nucleotides is a gap segment. In some embodiments, the oligonucleotide comprises at least one wing segment. In some embodiments, the oligonucleotide comprises at least one gap segment flanked by two wing segments. For example, the oligonucleotide comprises a gap segment flanked by a 5′-wing segment and a 3′-wing segment. In some embodiments, the gap segment or the wing segment comprises at least one chemical modification. In some embodiments, the antisense oligonucleotide is a gapmer.

In some embodiments, the oligonucleotide modulates a KRAS signaling pathway. In some embodiments, the KRAS signaling pathway comprises a KRAS-RAF-MEK-ERK signaling pathway. In some embodiments, the KRAS signaling pathway comprises phosphoinositide 3-kinase (PI3K) signaling pathway, mitogen-activated protein kinase (MAPK) signaling pathway, or Ral guanine nucleotide exchange factor (Ral-GEF) signaling pathway. As such, in some embodiments, the decreasing of the expression of the gene due to the binding of the oligonucleotide to the endogenous nucleic acid can further decrease a signaling pathway expression comprising the gene modulated by the oligonucleotide. In some embodiments, the decreasing gene or signaling pathway expression leads to therapeutic effects for treating the disease or condition. In some embodiments, the disease or condition is caused by increased gene or signaling pathway expression. In some embodiments, the disease or condition described herein is caused by genetic mutations associated with the gene or signaling pathway, such as mutated KRAS.

In aspects and embodiments, there is provided compounds comprising an antisense oligonucleotide that inhibits the expression of KRAS mRNA (including mRNA encoding mutated KRAS in some embodiments), wherein the antisense oligonucleotide comprises 10 to 30 linked nucleotides and has a sequence that is complementary to KRAS mRNA, and wherein the oligonucleotide has at least 8 contiguous nucleotides of any one of SEQ ID NOS: 1-10 and 14-21.

In embodiments, the oligonucleotide comprises at least 10, or at least 12, or at least 14 contiguous nucleobases of any one of SEQ ID NOs: 1-10 and 14-21. In embodiments, the oligonucleotide is from 10-24 nucleotides in length, such as from 10 to 16 or from 12 to 16 nucleotides in length. In embodiments, the oligonucleotide is 12, 13, 14, 15, or 16 nucleotides in length. For example, in embodiments the oligonucleotides is 13 or 14 nucleotides in length. In embodiments, the oligonucleotide comprises at least 12 contiguous nucleobases of any one of SEQ ID NOs: 1-10 and 14-21. In embodiments, the oligonucleotide comprises or consists of a nucleobase sequence of any one of SEQ ID NOs: 1-10 and 14-21. In embodiments, the oligonucleotide has a nucleobase sequence selected from SEQ ID NOs: 2, 3, 4, 5, 6, 7, 8, 9, and 10.

In some embodiments, the oligonucleotide described herein is an antisense oligonucleotide for targeting and binding (e.g., hybridizing) to an endogenous nucleic acid. In some embodiments, the binding of the oligonucleotide to the endogenous nucleic acid recruits endogenous nuclease (e.g., RNaseH) for degrading the endogenous nucleic acid. In some embodiments, the degradation of the endogenous nucleic acid decreases expression of the gene encoded by the endogenous nucleic acid. In some embodiments, the degradation of the endogenous nucleic acid can treat a disease or condition described herein.

In some embodiments, the oligonucleotide comprises 11 nucleic acid bases. In some embodiments, the oligonucleotide comprises 12 nucleic acid bases. In some embodiments, the oligonucleotide comprises 13 nucleic acid bases. In some embodiments, the oligonucleotide comprises 14 nucleic acid bases. In some embodiments, the oligonucleotide comprises 15 nucleic acid bases. In some embodiments, the oligonucleotide comprises 16 nucleic acid bases. In some embodiments, the oligonucleotide has no more than 20 nucleotides in length. In some embodiments, the oligonucleotide has no more than 19 nucleotides in length. In some embodiments, the oligonucleotide has no more than 18 nucleotides in length. In some embodiments, the oligonucleotide has no more than 17 nucleotides in length. In some embodiments, the oligonucleotide has no more than 16 nucleotides in length. In some embodiments, the oligonucleotide has no more than 15 nucleotides in length. In some embodiments, the oligonucleotide has no more than 14 nucleotides in length. In some embodiments, the oligonucleotide has no more than 13 nucleotides in length.

In some embodiments, the oligonucleotide comprises at least one gap segment. In some embodiments, the gap segment comprises four, five, six, seven, right, nine, 10, 11, 12, 13, or 14 nucleic acid bases. In some embodiments, the gap segment comprises four nucleic acid bases. In some embodiments, the gap segment comprises five nucleic acid bases. In some embodiments, the gap segment comprises six nucleic acid bases. In some embodiments, the gap segment comprises seven nucleic acid bases. In some embodiments, the gap segment comprises eight nucleic acid bases. In some embodiments, the gap segment comprises nine nucleic acid bases. In some embodiments, the gap segment comprises 10 nucleic acid bases. In some embodiments, the gap segment comprises 11 nucleic acid bases. In some embodiments, the gap segment comprises 12 nucleic acid bases. In some embodiments, the gap segment comprises 13 nucleic acid bases. In some embodiments, the gap segment comprises 14 nucleic acid bases. For example, in embodiments the antisense oligonucleotide has a stretch of at least 6 DNA nucleotides (a gap segment) sufficient to recruit RNaseH. In embodiments, the gap segment is 8 nucleotides.

In some embodiments, the oligonucleotide comprises at least one wing segment (e.g., a 5′ wing segment and a 3′ wing segment). In some embodiments, the at least one wing segment is a 5′-end wing segment that is covalently connected to the gap segment at the 5′-end of the gap segment. In some embodiments, the at least one wing segment is a 3′-end wing segment that is covalently connected to the gap segment at the 3′-end of the gap segment. In some embodiments, the gap segment is flanked by the wing segments at both the 5′-end and the 3′-end of the gap segment. In some embodiments, the wing segments independently comprise at least one, two, three, four, five, six, seven, eight, nine, 10, 11, 12, or more nucleic acid bases. In embodiments, the wing segments are independently from 2 to 6 nucleotides or from 2 to 4 nucleotides in length. In some embodiments, at least one wing segment comprises one nucleic acid base. In some embodiments, at least one wing segment comprises two nucleic acid bases. In some embodiments, at least one wing segment comprises three nucleic acid bases. In some embodiments, at least one wing segment comprises four nucleic acid bases. In some embodiments, at least one wing segment comprises five nucleic acid bases. In some embodiments, at least one wing segment comprises six nucleic acid bases. In some embodiments, at least one wing segment comprises seven nucleic acid bases. In embodiments, the length of the wing segments are independently selected from 2 to 5 nucleic acid bases. In embodiments, nucleotides in the wing segments comprise RNA nucleotides and/or 2′-O-modified nucleotides as described in detail herein.

In some embodiments, a KRAS mRNA sequence (e.g., encoded by a target cell) comprises any one of SEQ ID NOs: 11-13, 22 and 23. In some embodiments, wildtype and mutated KRAS mRNA sequences comprise any of those described in Table 4.

In embodiments, the oligonucleotide comprises at least 8 contiguous nucleobases of any one of SEQ ID NOs: 1-10 to 14-21, and the oligonucleotide is substantially (i.e., at least 90%) or entirely complementary to a segment of KRAS mRNA. In embodiments, the oligonucleotide comprises at least 10 or at least 12 contiguous nucleobases of any one of SEQ ID NOs: 1-10 to 14-21, and the oligonucleotide is substantially (i.e., at least 90%) or entirely complementary to a segment of KRAS mRNA. In certain embodiments, the oligonucleotide comprises or consists of a nucleobase sequence of any one of SEQ ID NOs: 1-10 and 14-21.

In embodiments, the oligonucleotide comprises at least 8 contiguous nucleobases of any one of SEQ ID NOs: 2, 3, 4, 5, 6, 7, 8, 9, and 10, and the oligonucleotide is substantially (i.e., at least 90%) or entirely complementary to a segment of KRAS mRNA. In embodiments, the oligonucleotide comprises at least 10 or at least 12 contiguous nucleobases of any one of SEQ ID NOs: 2, 3, 4, 5, 6, 7, 8, 9, and 10, and the oligonucleotide is substantially (i.e., at least 90%) or entirely complementary to a segment of KRAS mRNA. In certain embodiments, the oligonucleotide comprises or consists of a nucleobase sequence of any one of SEQ ID NOs: 2, 3, 4, 5, 6, 7, 8, 9, and 10.

For simplicity nucleotide sequences may be shown herein using DNA nucleotide sequences (i.e., including thymine nucleobases, “T” or “t”) or as RNA nucleotide sequences (i.e., including uracil nucleobases, “U” or “u”). It is understood from the context that when the nucleotide or sequence is intended to be RNA, T nucleotides can be substituted with U (or modified U such as pseudouridine or 1-methylpseudouridine); and when the nucleotide or sequence is intended to be DNA, U nucleotides can be substituted with T or modified T. However, in embodiments, RNA nucleotides in the antisense oligonucleotide may employ T (thymine) bases, and DNA nucleotides in the antisense oligonucleotides may employ U (uracil) bases. In some embodiments, one or more T nucleotides in the antisense oligonucleotides disclosed herein are swapped with U or modified U.

In some embodiments, the oligonucleotide has a nucleobase sequence (or chemical structure) selected from Tables 1-3, and 6-11. In embodiments, the oligonucleotide may have a nucleobase sequence selected from SEQ ID NOs: 1-10 and 14-21. In embodiments, the oligonucleotide may have a nucleobase sequence selected from SEQ ID NO: 2, 3, 4, 5, 6, 7, 8, 9, and 10.

In some embodiments, the binding (e.g., hybridization) of the antisense oligonucleotide to the target mRNA leads to degradation of the target mRNA or blocks translation of the target mRNA. In some embodiments, the binding of the antisense oligonucleotide to the target mRNA creates a duplex nucleic acid molecule, which then recruits an endogenous nuclease for degradation of the mRNA. In some embodiments, the antisense oligonucleotide has a stretch of DNA nucleotides sufficient to recruit RNaseH, and thereby trigger degradation of the target mRNA. In embodiments, the antisense oligonucleotide may have a stretch (e.g., a central stretch) of at least 6 or at least 8 DNA nucleotides, and which is optionally a stretch of 9 or 10 DNA nucleotides. In some embodiments, one or more DNA nucleotides comprise a 2′ chemical modification independently selected from 2′-Fluoro, 2′-Methyl, and 2′-Ethyl. In embodiments, DNA nucleotides do not comprise a 2′ chemical modification.

In embodiments, the antisense oligonucleotide may be a gapmer having a 5′ and a 3′ segment, each of the 5′ and 3′ segments being from 2 to 6 nucleotides or from 2 to 4 nucleotides, and where the 5′ and 3′ segments do not contain DNA nucleotides or do not contain only DNA nucleotides. In some embodiments, the gapmer is a 3-8-3 gapmer, having a central bock of DNA nucleotides and 5′ and 3′ segments of 3 RNA nucleotides each. In other embodiments, the gapmer is a 2-10-2 gapmer, having a central block of 10 DNA nucleotides and a 5′ and 3′ segments of 2 RNA nucleotides each. Other gapmer formats may be used, such as 3-10-2 and 2-10-3. In some embodiments, one or more nucleotides of the 5′ segment and the 3′ segment comprise 2′-O substituents, optionally where all of the nucleotides of the 5′ segment and the 3′ segment comprise 2′-O substituents. Exemplary 2′-O substituents are independently selected from 2′-O methyl, 2′-O ethyl, 2′-O methoxyethyl (MOE), and a bridged nucleotide (e.g., a locked or bi-cyclic nucleotide) having a 2′ to 4′ bridge. In some embodiments, the bridged nucleotide has a methylene bridge (LNA) or a constrained ethyl bridge (cEt).

The term “gapmer” refers to an oligonucleotide having a central block of deoxynucleotides (also referred to herein as “DNA nucleotides”) with 5′ and 3′ segments (of at least 2 nucleotides) comprising RNA nucleotides or 2′-O-modified nucleotides. As used herein, the term “DNA nucleotide” refers to a nucleotide that is not an RNA nucleotide. DNA nucleotides typically have a 2′ H, but may alternatively have various 2′ chemical modifications, including 2′-halo and 2′-lower alkyl (e.g., C1-4). In some embodiments, the 2′ chemical modifications of DNA nucleotides are independently selected from 2′-Fluoro, 2′-Methyl, and 2′-Ethyl.

Locked nucleic acid (LNA) or “locked nucleotides” are described, for example, in U.S. Pat. Nos. 6,268,490; 6,316,198; 6,403,566; 6,770,748; 6,998,484; 6,670,461; and 7,034,133, all of which are hereby incorporated by reference in their entireties. LNAs are modified nucleotides that contain a bridge between the 2′ and 4′ carbons of the sugar moiety resulting in a “locked” conformation, and/or bicyclic structure. Other suitable locked nucleotides that can be incorporated in the oligonucleotides of this disclosure include those described in U.S. Pat. Nos. 6,403,566 and 6,833,361, both of which are hereby incorporated by reference in their entireties. In exemplary embodiments, the locked nucleotides are independently selected from a 2′ to 4′ methylene bridge and a constrained ethyl (cEt) bridge (see, U.S. Pat. Nos. 7,399,845 and 7,569,686, which are hereby incorporated by reference in their entireties).

In some embodiments, the antisense oligonucleotide has a modified backbone or modified internucleotide linkages. The term “internucleotide linkage” refers to the linkage between two adjacent nucleosides in a polynucleotide molecule. Naturally, the internucleotide linkage is a phosphodiester bond that forms between two oxygen atoms of the phosphate group and an oxygen atom of the sugar (either at 3′ or 5′ position) to form two ester bonds bridging between the two adjacent nucleosides. Modification of the internucleotide linkage may provide different characteristics, including but not limited to enhanced stability. For example, phosphorothioate or phosphorodithioate linkages increase the resistance of the internucleotide linkage to nucleases. Another example is phosphoacetate linkage (PACE), which improves transfection characteristics and enhances nuclease resistance. Internucleotide linkages and oligonucleotide backbone modifications which may be employed in the oligonucleotides of the present description include, but are not limited to, phosphodiester, phosphorothioate, phosphorodithioate, methylphosphonate, alkylphosphonate, alkylphosphonothioate, phosphotriester, phosphoramidate, phosphoramidite, phosphorodiamidate, siloxane, carbonate, carboalkoxy, acetamidate, carbamate, morpholino, peptide nucleic acid, borano, thioether, bridged phosphoramidate, bridged methylene phosphonate, bridged phosphorothioate, and sulfone internucleoside linkages. In some embodiments, the antisense oligonucleotide comprises one or more phosphorothioate or phosphorodithioate nucleotides.

In some embodiments, the antisense oligonucleotide comprises one or more phosphorothioate or phosphorodithioate internucleotide linkages. In some embodiments, phosphorothioate or phosphorodithioate bonds can be introduced between the last three to five nucleotides at the 5′- and/or 3′-end of the oligonucleotide to reduce exonuclease degradation. In some embodiments, the antisense oligonucleotide has a combination of phosphodiester and phosphorothioate/phosphorodithioate linkages. In some embodiments, the antisense oligonucleotide contains at least two, at least three, at least four, at least five, at least six, at least seven, at least eight, at least nine, or at least ten phosphorothioate or phosphorodithioate internucleotide linkages. In some embodiments, the antisense oligonucleotide comprises substantially alternating phosphodiester and phosphorothioate internucleotide linkages. In some embodiments, backbone modifications described in this paragraph are used at least for internucleotide linkages for nucleotides in the wing segments. In some embodiments, the antisense oligonucleotide is fully phosphorothioate/phosphorodithioate linked (i.e., all bonds are either phosphorothioate or phosphorodithioate). In some embodiments, the ASO is fully phosphorothioate linked.

In some embodiments, particularly where RNaseH recruitment is not desired, the antisense oligonucleotide has a morpholino backbone. Morpholino oligonucleotides do not generally trigger the degradation of their target RNA molecules, and can be effective for steric blocking of a target RNA sequence. Morpholino oligonucleotides and their synthesis are disclosed generally in U.S. Pat. Nos. 11,028,386, 10,947,533, and 10,927,378, each of which is hereby incorporated by reference in its entirety. In some embodiments, the antisense oligonucleotide comprises thiomorpholino nucleotides and/or other substituted or modified nucleotides such as those described, for example, in International Patent Application Publication No. WO/2019/060522 and International Patent Application Publication No. WO/2018/057430, each of which is hereby incorporated by reference in its entirety. For example, Langner et al. describe methods for synthesizing oligonucleotide analogs dubbed thiophosphoramidate morpholino oligonucleotides (TMOs) which incorporate morpholino nucleosides and phosphorothioate linkages (“Synthesis and characterization of thiophosphoramidate morpholino oligonucleotides and chimeras.” JACS 142.38 (2020): 16240-16253; see also Dumbović, Gabrijela, et al. “Nuclear compartmentalization of TERT mRNA and TUG1 lncRNA is driven by intron retention.” Nature Communications 12.1 (2021): 1-19; both of which are hereby incorporated by reference in their entireties). Thus, the antisense oligonucleotides described herein may comprise full or partial TMO-modified nucleotides, or may comprise chimeras of TMO-modified nucleotides and unmodified nucleotides and/or other nucleotides comprising different modifications (e.g., LNAs).

In some embodiments, the antisense oligonucleotide may contain one or more modified bases. In some embodiments, cytosine is replaced with modified cytosine, such as 5-methylcytosine or 5-hydroxymethylcytosine, which may enhance base pairing. Other modified bases (particularly of cytosine or guanine) can be employed to reduce immunogenicity, where needed. Other modified bases are described in U.S. Pat. No. 10,064,959, which is hereby incorporated by reference. In various embodiments, cytidine nucleobases in the antisense oligonucleotide are 5-methyl cytidine. Thus, it will be understood by the skilled person that where a sequence includes a cytidine nucleobase (“C”), the term includes 5-methyl C. Further, it is understood that where uracil bases are included in a sequence, the term “U” includes pseudouridine and N1-methylpseudouridine.

In some embodiments, the oligonucleotide, upon binding to the target nucleic acid, forms a duplex with the target nucleic acid and recruits a nuclease for degrading the target nucleic acid. In some embodiments, the nuclease is a deoxyribonuclease. In some embodiments, the nuclease is a ribonuclease. In some embodiments, the ribonucleases is an endoribonuclease. In some embodiments, the endoribonuclease comprises endoribonuclease or RNase A, P, H, I, III, T1, T2, U2, V1, PhyM, or V. In some embodiments, the ribonuclease is an exoribonuclease. In some embodiments, the exoribonuclease comprises RNase PH, II, R, D, or T. In some embodiments, the nuclease comprises polynucleotide phosphorylase (PNPase), oligoribonuclease, exoribonuclease I, or exoribonuclease II. In some embodiments, the ribonuclease recruited by the oligonucleotide binding to the endogenous nucleic acid is RNase H.

In some embodiments, the oligonucleotide comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications. In some embodiments, the oligonucleotide comprises at least one gap segment comprising at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications. In some embodiments, the oligonucleotide comprises at least one wing segment comprising at least one, two, three, four, five, six, seven, eight, nine, 10, 11, 12, 13, 14, 15, 16, 17, 18, 19, 20, 21, 22, 23, 24, 25, 26, 27, 28, 29, 30 or more chemical modifications. In some embodiments, the oligonucleotide comprises at least one gap segment and at least one wing segment comprising at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications.

In some embodiments, the oligonucleotide described herein binds to a nucleic acid (e.g., a mRNA) encoding KRAS, where the binding of the oligonucleotide to the KRAS nucleic acid encoding KRAS (e.g., KRAS mRNA) decreases the nucleic acid encoding KRAS in a cell by at least 10%, 20%, 30%, 40%, 50%, 60%, 70%, 80%, 90%, or more compared to the expression of KRAS not modulated by the oligonucleotide. In some embodiments, the oligonucleotide described herein binds to a nucleic acid (e.g., a mRNA) encoding mutated KRAS, where the binding of the oligonucleotide to the nucleic acid encoding mutated KRAS decreases the endogenous expression of mutated KRAS in a cell by at least 10%, 20%, 30%, 40%, 50%, 60%, 70%, 80%, 90%, or more compared to the expression of mutated KRAS not modulated by the oligonucleotide. In some embodiments, the mutated KRAS nucleic acid encodes a mutated KRAS protein comprising a G12C mutation, a G12V mutation, a G12A mutation, or a G12D mutation.

In some embodiments, the oligonucleotide described herein binds to a nucleic acid (e.g., a mRNA) encoding KRAS, where the binding of the oligonucleotide to the KRAS decreases the activity or expression of molecules in or associated with the KRAS-RAF-MEK-ERK signaling pathway, the PI3K signaling pathway, the MAPK signaling pathway, or the Ral-GEF signaling pathway in a cell by at least 10%, 20%, 30%, 40%, 50%, 60%, 70%, 80%, 90%, or more compared to the activity or expression of molecules in or associated with of the KRAS-RAF-MEK-ERK signaling pathway, the PI3K signaling pathway, the MAPK signaling pathway, or the Ral-GEF signaling pathway not modulated by the oligonucleotide.

In some embodiments, the oligonucleotide described herein binds to a nucleic acid (e.g., a mRNA) encoding mutated KRAS, where the binding of the oligonucleotide to the nucleic acids encoding mutated KRAS decreases the endogenous expression of a gene in, or an activity of, the KRAS-RAF-MEK-ERK signaling pathway, the PI3K signaling pathway, the MAPK signaling pathway, or the Ral-GEF signaling pathway in a cell by at least 10%, 20%, 30%, 40%, 50%, 60%, 70%, 80%, 90%, or more compared to the expression of a gene in, or an activity of the KRAS-RAF-MEK-ERK signaling pathway, the PI3K signaling pathway, the MAPK signaling pathway, or the Ral-GEF signaling pathway not modulated by the oligonucleotide.

In some embodiments, the compound comprises at least two oligonucleotides, where a first oligonucleotide binds to a nucleic acid (e.g., a mRNA) encoding a KRAS and a second oligonucleotide binds to another nucleic acid encoding a mutated KRAS. In some embodiments, the nucleic acid encoding the mutated KRAS encodes a mutated KRAS protein comprising a G12C mutation, a G12V mutation, a G12A mutation, or a G12D mutation. In some embodiments, the compound comprises at least two oligonucleotides, where a first oligonucleotide binds to a nucleic acid (e.g., a mRNA) encoding a first mutated KRAS and a second oligonucleotide binds to another nucleic acid encoding a second mutated KRAS, wherein the first and second mutated KRAS are different. The two different mutated KRAS may have different amino acid sequences. In some embodiments, the two first and second mutated KRAS may be the same, but the nucleic acids encoding them have different sequences. In some embodiments, the compound comprises at least two oligonucleotides, where a first oligonucleotide and a second oligonucleotide bind to a nucleic acid encoding another a KRAS.

In some embodiments, the binding of the oligonucleotides to both KRAS and mutated KRAS decreases the endogenous expression of a gene in, or an activity of, the KRAS-RAF-MEK-ERK signaling pathway, the PI3K signaling pathway, the MAPK signaling pathway, or the Ral-GEF signaling pathway in a cell by at least 10%, 20%, 30%, 40%, 50%, 60%, 70%, 80%, 90%, or more compared to the endogenous expression of a gene in, or an activity of the KRAS-RAF-MEK-ERK signaling pathway, the PI3K signaling pathway, the MAPK signaling pathway, or the Ral-GEF signaling pathway not modulated by the oligonucleotide.

In some embodiments, the compound is formulated for administration to a subject by appropriate administration routes, including but not limited to, intravenous, intratumoral, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations. In some embodiments, the compound is formulated into a dosage form. In some embodiments, the compound is formulated to include at least one excipient. In some embodiments, the excipient is a pharmaceutically acceptable excipient.

In some embodiments, the compound comprising the oligonucleotide described herein treats a disease or condition by decreasing the expression of the gene or the signaling pathway associated with the disease or condition. In some embodiments, the compound comprising the oligonucleotide describe herein treats the disease or condition described herein by directly decreasing the gene expression associated with disease or condition described herein. In some embodiments, the compound comprising the oligonucleotide treats the disease or condition by decreasing the gene expression as part of a signaling pathway described herein. In some embodiments, the compound comprising the oligonucleotide described herein treats a disease or condition by decreasing the KRAS expression (e.g., mutated KRAS expression). In some embodiments, the compound comprising the oligonucleotide described herein treats a disease or condition by decreasing the KRAS expression. In some embodiments, the compound comprising the oligonucleotide described herein treats a disease or condition by decreasing both KRAS and mutated KRAS expressions. In some embodiments, the compound comprising the oligonucleotide described herein treats a disease or condition by decreasing the endogenous KRAS expression. In some embodiments, the compound comprising the oligonucleotide described herein treats a disease or condition by decreasing the endogenous the KRAS-RAF-MEK-ERK signaling pathway, the PI3K signaling pathway, the MAPK signaling pathway, or the Ral-GEF signaling pathway expression or activity. In some embodiments, the disease or condition described herein is cancer.

In some embodiments, the chemical modifications of the oligonucleotide comprise the chemical modifications listed in Table 1-3, and 6-11. In some embodiments, the oligonucleotide is an oligonucleotide listed in Table 1-3 and 6-11.

In some embodiments, chemical modification can occur at 3′-OH group, 5′-OH group, at the backbone, at the sugar component, or at the nucleotide base. Chemical modification can include non-naturally occurring linker molecules of interstrand or intrastrand cross links. In one aspect, the chemically modified nucleic acid comprises modification of one or more of the 3′-OH or 5′-OH group, the backbone, the sugar component, or the nucleotide base, or addition of non-naturally occurring linker molecules. In some embodiments, chemically modified backbone comprises a backbone other than a phosphodiester backbone. In some embodiments, a modified sugar comprises a sugar other than deoxyribose (in modified DNA) or other than ribose (modified RNA). In some embodiments, a modified base comprises a base other than adenine, guanine, cytosine, thymine or uracil. In some embodiments, the oligonucleotide comprises at least one chemically modified base. In some instances, the comprises at least one, two, three, four, five, six, seven, eight, nine, 10, 15, 20, or more modified bases. In some embodiments, chemical modifications to the base moiety include natural and synthetic modifications of adenine, guanine, cytosine, thymine, or uracil, and purine or pyrimidine bases.

In some embodiments, the at least one chemical modification of the oligonucleotide comprises a modification of any one of or any combination of: 2′ modified nucleotide comprising 2′-O-methyl, 2′-O-methoxyethyl(2′-O-MOE), 2′-O-aminopropyl, 2′-deoxy, 2′-deoxy-2′-fluoro, 2′-O-aminopropyl(2′-O-AP), 2′-O-dimethylaminoethyl(2′-O-DMAOE), 2′-O-dimethylaminopropyl(2′-O-DMAP), 2′-O-dimethylaminoethyloxyethyl(2′-O-DMAEOE), or 2′-O-N-methylacetamido (2′-O-NMA); modification of one or both of the non-linking phosphate oxygens in the phosphodiester backbone linkage; modification of one or more of the linking phosphate oxygens in the phosphodiester backbone linkage; modification of a constituent of the ribose sugar; replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Non limiting examples of chemical modification to the oligonucleotide can include: modification of one or both of non-linking or linking phosphate oxygens in the phosphodiester backbone linkage (e.g., sulfur(S), selenium (Se), BR3 (wherein R can be, e.g., hydrogen, alkyl, or aryl), C (e.g., an alkyl group, an aryl group, and the like), H, NR2, wherein R can be, e.g., hydrogen, alkyl, or aryl, or wherein R can be, e.g., alkyl or aryl); replacement of the phosphate moiety with “dephospho” linkers (e.g., replacement with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino); modification or replacement of a naturally occurring nucleobase with nucleic acid analog; modification of deoxyribose-phosphate or ribose-phosphate backbone (e.g., modifying the ribose-phosphate backbone to incorporate phosphorothioate, phosphonothioacetate, phosphoroselenates, boranophosphates, borano phosphate esters, hydrogen phosphonates, phosphonocarboxylate, phosphoroamidates, alkyl or aryl phosphonates, phosphonoacetate, or phosphotriesters; modification of 5′ end (e.g., 5′ cap or modification of 5′ cap-OH) or 3′ end of the nucleic acid sequence (3′ tail or modification of 3′ end-OH); substitution of the phosphate group with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino; modification of the ribophosphate backbone to incorporate morpholino (phosphorodiamidate morpholino oligomer PMO), thiomorpholino, cyclobutyl, pyrrolidine, or peptide nucleic acid (PNA) nucleoside surrogates; modifications to the sugar of a nucleotide to incorporate locked nucleic acid (LNA), unlocked nucleic acid (UNA), ethylene nucleic acid (ENA), constrained ethyl (cEt) sugar, or bridged nucleic acid (BNA); modification of a constituent of the ribose sugar (e.g., 2′-O-methoxyethoxy (2′-MOEr), 2′-O-methyl, 2′-O-methoxy-ethyl(2′-O-MOE), 2′-fluoro, 2′-aminoethyl, 2′-deoxy-2′-fuloarabinou-cleic acid, 2′-deoxy, 2′-O-methyl, 3′-phosphorothioate, 3′-phosphonoacetate (PACE), or 3′-phosphonothioacetate (thioPACE)); modification to the base of a nucleotide (of A, T, C, G, or U; for example, a 5-hydroxymethyl modified nucleotide such as a 5-Hydroxymethylcytosine); and stereopure of nucleotide (e.g., S conformation of phosphorothioate or R conformation of phosphorothioate).

In some embodiments, the chemical modification of the oligonucleotide comprises at least one substitution of one or both of non-linking phosphate oxygen atoms in a phosphodiester backbone linkage of the oligonucleotide. In some embodiments, the at least one chemical modification of the oligonucleotide comprises a substitution of one or more of linking phosphate oxygen atoms in a phosphodiester backbone linkage of the oligonucleotide. A non-limiting example of a chemical modification of a phosphate oxygen atom is a sulfur atom. In some embodiments, the chemical modification of the oligonucleotide comprises at least one chemical modification to a sugar of a nucleotide of the oligonucleotide. In some embodiments, the chemical modification of the oligonucleotide comprises at least one chemical modification to the sugar of the nucleotide, where the chemical modification comprises at least one locked nucleic acid (LNA). In some embodiments, the chemical modification of the oligonucleotide comprises at least one chemical modification to the sugar of the nucleotide of the oligonucleotide comprising at least one unlocked nucleic acid (UNA). In some embodiments, the chemical modification of the oligonucleotide comprises at least one chemical modification to the sugar of the nucleotide of the oligonucleotide comprising at least one ethylene nucleic acid (ENA). In some embodiments, the chemical modification of the oligonucleotide comprises at least one chemical modification to the sugar comprising a modification of a constituent of the sugar, where the sugar is a ribose sugar. In some embodiments, the chemical modification of the oligonucleotide comprises at least one chemical modification to the constituent of the ribose sugar of the nucleotide of the oligonucleotide comprising a 2′-O-methyl group. In some embodiments, the chemical modification of the oligonucleotide comprises at least one chemical modification comprising replacement of a phosphate moiety of the oligonucleotide with a dephospho linker. In some embodiments, the chemical modification of the oligonucleotide comprises at least one chemical modification of a phosphate backbone of the oligonucleotide. In some embodiments, the oligonucleotide comprises a phosphorothioate group. In some embodiments, the chemical modification of the oligonucleotide comprises at least one chemical modification comprising a modification to a base of a nucleotide of the oligonucleotide. In some embodiments, the chemical modification of the oligonucleotide comprises at least one chemical modification comprising an unnatural base of a nucleotide. In some embodiments, the chemical modification of the oligonucleotide comprises at least one chemical modification comprising a morpholino group (e.g., a phosphorodiamidate morpholino oligomer, PMO), a cyclobutyl group, pyrrolidine group, or peptide nucleic acid (PNA) nucleoside surrogate. In some embodiments, the chemical modification of the oligonucleotide comprises at least one chemical modification comprising at least one stereopure nucleic acid. In some embodiments, the at least one chemical modification can be positioned proximal to a 5′ end of the oligonucleotide. In some embodiments, the at least one chemical modification can be positioned proximal to a 3′ end of the oligonucleotide. In some embodiments, the at least one chemical modification can be positioned proximal to both 5′ and 3′ ends of the oligonucleotide.

In some embodiments, an oligonucleotide comprises a backbone comprising a plurality of sugar and phosphate moieties covalently linked together. In some embodiments, a backbone of an oligonucleotide comprises a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some embodiments, a backbone of an oligonucleotide can lack a 5′ reducing hydroxyl, a 3′ reducing hydroxyl, or both, capable of being exposed to a solvent. In some embodiments, a backbone of an oligonucleotide can lack a 5′ reducing hydroxyl, a 3′ reducing hydroxyl, or both, capable of being exposed to nucleases. In some embodiments, a backbone of an oligonucleotide can lack a 5′ reducing hydroxyl, a 3′ reducing hydroxyl, or both, capable of being exposed to hydrolytic enzymes. In some instances, a backbone of an oligonucleotide can be represented as a polynucleotide sequence in a circular 2-dimensional format with one nucleotide after the other. In some instances, a backbone of an oligonucleotide can be represented as a polynucleotide sequence in a looped 2-dimensional format with one nucleotide after the other. In some embodiments, a 5′ hydroxyl, a 3′ hydroxyl, or both, are joined through a phosphorus-oxygen bond. In some embodiments, a 5′ hydroxyl, a 3′ hydroxyl, or both, are modified into a phosphoester with a phosphorus-containing moiety.

In some embodiments, the oligonucleotide described herein comprises at least one chemical modification. A chemical modification can be a substitution, insertion, deletion, chemical modification, physical modification, stabilization, purification, or any combination thereof. In some embodiments, a modification is a chemical modification. Suitable chemical modifications comprise any one of: 5′-adenylate, 5′-guanosine-triphosphate cap, 5′-N7-Methylguanosine-triphosphate cap, 5′-triphosphate cap, 3′-phosphate, 3′-thiophosphate, 5′-phosphate, 5′-thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′-DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphorothioate DNA, phosphorothioate RNA, UNA, LNA, cEt, pseudouridine-5′-triphosphate, 5′-methylcytidine-5′-triphosphate, 2′-O-methyl-phosphorothioate or any combinations thereof.

In some embodiments, the oligonucleotide modification can alter physio-chemical properties of a nucleotide, such as their conformation, polarity, hydrophobicity, chemical reactivity, base-pairing interactions, or any combination thereof. A chemical modification can also be a phosphorothioate substitute. In some embodiments, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some embodiments, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases is of high probability in vivo or in vitro. For example, phosphorothioate (PS) bonds can be introduced between the last 3-5 nucleotides at the 5′- or 3′-end of a polynucleic acid which can inhibit exonuclease degradation. In some embodiments, phosphorothioate bonds can be added throughout an entire polynucleic acid to reduce attack by endonucleases. In some embodiments, the oligonucleotide described herein comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 50, 100, or more internucleotide linkage comprising PS bond. In some embodiments, the oligonucleotide described herein comprises only PS bond as the internucleotide linkage modification. In some embodiments, all internucleotide linkages of the oligonucleotide described herein are fully PS-modified or include phosphorothioate internucleotide linkages. In some embodiments, the oligonucleotide comprising PS bond as the internucleotide linkage modification comprises a 5′-end wing segment comprising one nucleic acid base. In some embodiments, the oligonucleotide comprising PS bond as the internucleotide linkage modification comprises a 5′-end wing segment comprising two nucleic acid bases. In some embodiments, the oligonucleotide comprising PS bond as the internucleotide linkage modification comprises a 5′-end wing segment comprising three nucleic acid bases. In some embodiments, the oligonucleotide comprising PS bond as the internucleotide linkage modification comprises a 5′-end wing segment comprising four nucleic acid bases. In some embodiments, the oligonucleotide comprising PS bond as the internucleotide linkage modification comprises a 5′-end wing segment comprising five nucleic acid bases. In some embodiments, the oligonucleotide comprising PS bond as the internucleotide linkage modification comprises a 5′-end wing segment comprising six nucleic acid bases. In some embodiments, the oligonucleotide comprising PS bond as the internucleotide linkage modification comprises a 5′-end wing segment comprising seven nucleic acid bases. In some embodiments, the oligonucleotide comprising PS bond as the internucleotide linkage modification comprises a 5′-end wing segment comprising eight nucleic acid bases. In some embodiments, the oligonucleotide comprising PS bond as the internucleotide linkage modification comprises a 5′-end wing segment comprising nine nucleic acid bases. In some embodiments, the oligonucleotide comprising PS bond as the internucleotide linkage modification comprises a 5′-end wing segment comprising 10 nucleic acid bases. In some embodiments, the oligonucleotide comprising PS bond as the internucleotide linkage modification comprises a 3′-end wing segment comprising one nucleic acid base. In some embodiments, the oligonucleotide comprising PS bond as the internucleotide linkage modification comprises a 3′-end wing segment comprising two nucleic acid bases. In some embodiments, the oligonucleotide comprising PS bond as the internucleotide linkage modification comprises a 3′-end wing segment comprising three nucleic acid bases. In some embodiments, the oligonucleotide comprising PS bond as the internucleotide linkage modification comprises a 3′-end wing segment comprising four nucleic acid bases. In some embodiments, the oligonucleotide comprising PS bond as the internucleotide linkage modification comprises a 3′-end wing segment comprising five nucleic acid bases. In some embodiments, the oligonucleotide comprising PS bond as the internucleotide linkage modification comprises a 3′-end wing segment comprising six nucleic acid bases. In some embodiments, the oligonucleotide comprising PS bond as the internucleotide linkage modification comprises a 3′-end wing segment comprising seven nucleic acid bases. In some embodiments, the oligonucleotide comprising PS bond as the internucleotide linkage modification comprises a 3′-end wing segment comprising eight nucleic acid bases. In some embodiments, the oligonucleotide comprising PS bond as the internucleotide linkage modification comprises a 3′-end wing segment comprising nine nucleic acid bases. In some embodiments, the oligonucleotide comprising PS bond as the internucleotide linkage modification comprises a 3′-end wing segment comprising 10 nucleic acid bases.

In some embodiments, the oligonucleotide comprising PS bond as the internucleotide linkage modification comprises a 5′-end wing segment comprising one nucleic acid base and a 3′-end wing segment comprising one nucleic acid base. In some embodiments, the oligonucleotide comprising PS bond as the internucleotide linkage modification comprises a 5′-end wing segment comprising two nucleic acid bases and a 3′-end wing segment comprising two nucleic acid bases. In some embodiments, the oligonucleotide comprising PS bond as the internucleotide linkage modification comprises a 5′-end wing segment comprising three nucleic acid bases and a 3′-end wing segment comprising three nucleic acid bases. In some embodiments, the oligonucleotide comprising PS bond as the internucleotide linkage modification comprises a 5′-end wing segment comprising four nucleic acid bases and a 3′-end wing segment comprising four nucleic acid bases. In some embodiments, the oligonucleotide comprising PS bond as the internucleotide linkage modification comprises a 5′-end wing segment comprising five nucleic acid bases and a 3′-end wing segment comprising five nucleic acid bases.

In some embodiments, the oligonucleotide comprises a 5′-end wing segment comprising one nucleic acid base, a gapmer, and a 3′-end wing segment comprising one nucleic acid base, where the internucleotide linkages of the oligonucleotide joining the 5′-end wing segment, the gapmer, and the 3′-end wing segment comprises only PS bonds. In some embodiments, the oligonucleotide comprises a 5′-end wing segment comprising two nucleic acid bases, a gapmer, and a 3′-end wing segment comprising two nucleic acid bases, where the internucleotide linkages of the oligonucleotide joining the 5′-end wing segment, the gapmer, and the 3′-end wing segment comprises only PS bonds. In some embodiments, the oligonucleotide comprises a 5′-end wing segment comprising three nucleic acid bases, a gapmer, and a 3′-end wing segment comprising three nucleic acid bases, where the internucleotide linkages of the oligonucleotide joining the 5′-end wing segment, the gapmer, and the 3′-end wing segment comprises only PS bonds. In some embodiments, the oligonucleotide comprises a 5′-end wing segment comprising four nucleic acid bases, a gapmer, and a 3′-end wing segment comprising four nucleic acid bases, where the internucleotide linkages of the oligonucleotide joining the 5′-end wing segment, the gapmer, and the 3′-end wing segment comprises only PS bonds. In some embodiments, the oligonucleotide comprises a 5′-end wing segment comprising five nucleic acid bases, a gapmer, and a 3′-end wing segment comprising five nucleic acid bases, where the internucleotide linkages of the oligonucleotide joining the 5′-end wing segment, the gapmer, and the 3′-end wing segment comprises only PS bonds. In some embodiments, the oligonucleotide comprises a 5′-end wing segment comprising six nucleic acid bases, a gapmer, and a 3′-end wing segment comprising six nucleic acid bases, where the internucleotide linkages of the oligonucleotide joining the 5′-end wing segment, the gapmer, and the 3′-end wing segment comprises only PS bonds.

In some embodiments, the oligonucleotide comprising the 5′-end wing segment, a gapmer, the 3′-end wing segment, and PS bond as internucleotide linkage comprises a nucleic acid sequence that is at least 70%, 75%, 80%, 85%, 90%, 95%, or 99% identical to SEQ ID NOs: 1-10, and 14-21. In some embodiments, the oligonucleotide comprising the 5′-end wing segment, a gapmer, the 3′-end wing segment, and PS bond as internucleotide linkage comprises a nucleic acid sequence that is at least 70%, 75%, 80%, 85%, 90%, 95%, or 99% identical to SEQ ID NOs: 2, 3, 4, 5, 6, 7, 8, 9, and 10. In some embodiments, the oligonucleotide comprising the 5′-end wing segment, a gapmer, the 3′-end wing segment, and PS bond as internucleotide linkage comprises a nucleic acid sequence that is at least 70%, 75%, 80%, 85%, 90%, 95%, or 99% identical to any sequence listed in Table 1-3, and 6-11.

An oligonucleotide can be circular, substantially circular, or otherwise linked in a contiguous fashion (e.g. can be arranged as a loop) and can also retain a substantially similar secondary structure as a substantially similar oligonucleotide that may not be circular or may not be a loop.

In some embodiments, the chemical modification comprises modification of one or both of the non-linking phosphate oxygens in the phosphodiester backbone linkage or modification of one or more of the linking phosphate oxygens in the phosphodiester backbone linkage. As used herein, “alkyl” is meant to refer to a saturated hydrocarbon group which is straight-chained or branched. Example alkyl groups include methyl (Me), ethyl (Et), propyl (e.g., n-propyl or isopropyl), butyl (e.g., n-butyl, isobutyl, or t-butyl), or pentyl (e.g., n-pentyl, isopentyl, or neopentyl). An alkyl group can contain from 1 to about 20, from 2 to about 20, from 1 to about 12, from 1 to about 8, from 1 to about 6, from 1 to about 4, or from 1 to about 3 carbon atoms. As used herein, “aryl” refers to monocyclic or polycyclic (e.g., having 2, 3, or 4 fused rings) aromatic hydrocarbons such as, for example, phenyl, naphthyl, anthracenyl, phenanthrenyl, indanyl, or indenyl. In some embodiments, aryl groups have from 6 to about 20 carbon atoms. As used herein, “alkenyl” refers to an aliphatic group containing at least one double bond. As used herein, “alkynyl” refers to a straight or branched hydrocarbon chain containing 2-12 carbon atoms and characterized in having one or more triple bonds. Examples of alkynyl groups can include ethynyl, propargyl, or 3-hexynyl. “Arylalkyl” or “aralkyl” refers to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. “Cycloalkyl” refers to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl. “Heterocyclyl” refers to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl. “Heteroaryl” refers to a monovalent radical of a heteroaromatic ring system. Examples of heteroaryl moieties can include imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

In some embodiments, the phosphate group of a chemically modified nucleotide can be modified by replacing one or more of the oxygens with a different substituent. In some embodiments, the chemically modified nucleotide can include replacement of an unmodified phosphate moiety with a modified phosphate as described herein. In some embodiments, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur(S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way is a stereogenic center. The stereogenic phosphorous atom can possess either the “R” configuration (herein Rp) or the “S” configuration (herein Sp). In some embodiments, the oligonucleotide comprises stereopure nucleotides comprising S conformation of phosphorothioate or R conformation of phosphorothioate. In some embodiments, the chiral phosphate product is present in a diastereomeric excess of 50%, 60%, 70%, 80%, 90%, or more. In some embodiments, the chiral phosphate product is present in a diastereomeric excess of 95%. In some embodiments, the chiral phosphate product is present in a diastereomeric excess of 96%. In some embodiments, the chiral phosphate product is present in a diastereomeric excess of 97%. In some embodiments, the chiral phosphate product is present in a diastereomeric excess of 98%. In some embodiments, the chiral phosphate product is present in a diastereomeric excess of 99%. In some embodiments, both non-bridging oxygens of phosphorodithioates can be replaced by sulfur. The phosphorus center in the phosphorodithioates can be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl). In some embodiments, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O) (NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and can be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like.

In some embodiments, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos. A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. It is also understood in a nucleotide substitute that both the sugar and the phosphate moieties of the nucleotide can be replaced, by for example an amide type linkage (aminoethylglycine) (PNA). It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S-H-phosphonate, a polyamine or a polyethylene glycol chain, or adamantane acetic acid, a palmityl moiety, or an octadecylamine or hexylamino-carbonyl-oxycholesterol moiety.

In some embodiments, the chemical modification described herein comprises modification of a phosphate backbone. In some embodiments, the oligonucleotide described herein comprises at least one chemically modified phosphate backbone. Exemplary chemically modification of the phosphate group or backbone can include replacing one or more of the oxygens with a different substituent. Furthermore, the modified nucleotide present in the oligonucleotide can include the replacement of an unmodified phosphate moiety with a modified phosphate as described herein. In some embodiments, the modification of the phosphate backbone can include alterations resulting in either an uncharged linker or a charged linker with unsymmetrical charge distribution. Exemplary modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur(S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp). In such case, the chemically modified oligonucleotide can be stereopure (e.g. S or R confirmation). In some embodiments, the chemically modified oligonucleotide comprises stereopure phosphate modification. For example, the chemically modified oligonucleotide comprises S conformation of phosphorothioate or R conformation of phosphorothioate.

Phosphorodithioates have both non-bridging oxygens replaced by sulfur. The phosphorus center in the phosphorodithioates is achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

In some embodiments, at least one phosphate group of the oligonucleotide can be chemically modified. In some embodiments, the phosphate group can be replaced by non-phosphorus containing connectors. In some embodiments, the phosphate moiety can be replaced by dephospho linker. In some embodiments, the charge phosphate group can be replaced by a neutral group. In some embodiments, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In some embodiments, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g., 3′-amino phosphoramidate and aminoalkylphosphoramidates), thionophosphoramidates, thionoalkylphosphonates, thionoalkylphosphotriesters, and boranophosphates. The phosphate or modified phosphate linkage between two nucleotides can be through a 3′-5′ linkage or a 2′-5′ linkage, and the linkage contains inverted polarity such as 3′-5′ to 5′-3′ or 2′-5′ to 5′-2′.

In some embodiments, the chemical modification described herein comprises modification by replacement of a phosphate group. In some embodiments, the oligonucleotide described herein comprises at least one chemically modification comprising a phosphate group substitution or replacement. Exemplary phosphate group replacement can include non-phosphorus containing connectors. In some embodiments, the phosphate group substitution or replacement can include replacing charged phosphate group can by a neutral moiety. Exemplary moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

In some embodiments, the chemical modification described herein comprises modifying ribophosphate backbone of the oligonucleotide. In some embodiments, the oligonucleotide described herein comprises at least one chemically modified ribophosphate backbone. Exemplary chemically modified ribophosphate backbone can include scaffolds that can mimic nucleic acids can also be constructed wherein the phosphate linker and ribose sugar are replaced by nuclease resistant nucleoside or nucleotide surrogates. In some embodiments, the nucleobases can be tethered by a surrogate backbone. Examples can include morpholino such as a phosphorodiamidate morpholino oligomer (PMO), cyclobutyl, pyrrolidine and peptide nucleic acid (PNA) nucleoside surrogates.

In some embodiments, the chemical modification described herein comprises modification of sugar. In some embodiments, the oligonucleotide described herein comprises at least one chemically modified sugar. Exemplary chemically modified sugar can include 2′ hydroxyl group (OH) modified or replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR, wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the “oxy”-2′ hydroxyl group modification can include (LNA, in which the 2′ hydroxyl can be connected, e.g., by a Ci-₆ alkylene or Cj-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the “oxy”-2′ hydroxyl group modification can include the methoxyethyl group (MOE), (OCH₂CH₂OCH₃, e.g., a PEG derivative). In some embodiments, the deoxy modifications can include hydrogen (i.e., deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein),NHC (O) R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the Γ position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that are in the L form, e.g. L-nucleosides. In some embodiments, the oligonucleotide described herein includes the sugar group ribose, which is a 5-membered ring having an oxygen. Exemplary modified nucleosides and modified nucleotides can include replacement of the oxygen in ribose (e.g., with sulfur(S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some embodiments, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid. In some embodiments, the modifications to the sugar of the oligonucleotide comprises modifying the oligonucleotide to include locked nucleic acid (LNA), unlocked nucleic acid (UNA), ethylene nucleic acid (ENA), constrained ethyl (cEt) sugar, or bridged nucleic acid (BNA).

In some embodiments, the oligonucleotide described herein comprises at least one chemical modification of a constituent of the ribose sugar. In some embodiments, the chemical modification of the constituent of the ribose sugar can include 2′-O-methyl, 2′-O-methoxyethyl(2′-O-MOE), 2′-fluoro, 2′-aminoethyl, 2′-deoxy-2′-fuloarabinou-cleic acid, 2′-deoxy, 2′-deoxy-2′-fluoro, 2′-O-methyl, 3′-phosphorothioate, 2′-O-aminopropyl(2′-O-AP), 2′-O-dimethylaminoethyl(2′-O-DMAOE), 2′-O-dimethylaminopropyl(2′-O-DMAP), 2′-O-dimethylaminoethyloxyethyl(2′-O-DMAEOE), 2′-O—N-methylacetamido (2′-O-NMA) 3′-phosphonoacetate (PACE), or 3′-phosphonothioacetate (thioPACE). In some embodiments, the chemical modification of the constituent of the ribose sugar comprises unnatural nucleic acid. In some instances, the unnatural nucleic acids include modifications at the 5′-position and the 2′-position of the sugar ring, such as 5′-CH₂-substituted 2′-O-protected nucleosides. In some embodiments, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃. Unnatural nucleic acids can include 2′-substituted 5′-CH₂ (or O) modified nucleosides. Unnatural nucleic acids can include 5′-methylenephosphonate DNA and RNA monomers, and dimers. Unnatural nucleic acids can include 5′-phosphonate monomers having a 2′-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof).

In some embodiments, unnatural nucleic acids also include modifications of the sugar moiety. In some embodiments, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R₁)(R₂) (R═H, C₁-C₁₂ alkyl or a protecting group); and combinations thereof.

In some instances, the oligonucleotide described herein comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol-like ribonucleosides.

Modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O—, S-or N-alkynyl; or O-alkyl-O-alkyl, wherein the alkyl, alkenyl and alkynyl can be substituted or unsubstituted C₁ to C₁₀, alkyl or C₂ to C₁₀ alkenyl and alkynyl, 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)n CH₃)]₂, where n and m are from 1 to about 10. Other chemical modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Chemically modified sugars also include those that contain modifications at the bridging ring oxygen, such as CH₂ and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O—2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof; 4′-C(CH₃) (CH₃)—O-2′ and analogs thereof.

In some embodiments, the chemical modification described herein comprises modification of the base of nucleotide (e.g. the nucleobase). Exemplary nucleobases can include adenine (A), thymine (T), guanine (G), cytosine (C), and uracil (U). These nucleobases can be modified or replaced to in the oligonucleotide described herein. The nucleobase of the nucleotide can be independently selected from a purine, a pyrimidine, a purine or pyrimidine analog. In some embodiments, the nucleobase can be naturally-occurring or synthetic derivatives of a base. In embodiments, nucleotide sequences may be shown herein using DNA nucleotide sequences (i.e., including thymine nucleobases, “T” or “t”) or as RNA nucleotide sequences (i.e., including uracil nucleobases, “U” or “u”). It is understood from the context that when the nucleotide or sequence is intended to be RNA, T nucleotides can be substituted with U (or modified U such as pseudouridine or 1-methylpseudouridine); and when the nucleotide or sequence is intended to be DNA, U nucleotides can be substituted with T or modified T. However, in embodiments, RNA nucleotides in the antisense oligonucleotide may employ T (thymine) bases, and DNA nucleotides in the antisense oligonucleotides may employ U (uracil) bases. In some embodiments, one or more T nucleotides in the antisense oligonucleotides disclosed herein are swapped with U or modified U.

In some embodiments, the chemical modification described herein comprises modifying an uracil. In some embodiments, the oligonucleotide described herein comprises at least one chemically modified uracil. Exemplary chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine, 1 methyl-pseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroundine, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropy pseudouridine, 5-(isopentenylaminomethyl) uridine, 5-(isopentenylaminomethyl)-2-thio-uridine, a-thio-uridine, 2′-O—methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino) uridine, pyrazolo[3,4-d]pyrimidines, xanthine, and hypoxanthine.

In some embodiments, the chemical modification described herein comprises modifying a cytosine. In some embodiments, the oligonucleotide described herein comprises at least one chemically modified cytosine. Exemplary chemically modified cytosine can include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, a-thio-cytidine, 2′-O-methyl-cytidine, 5,2′-O-dimethyl-cytidine, N4-acetyl-2′-O-methyl-cytidine, N4,2′-O—dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some embodiments, the chemical modification described herein comprises modifying a adenine. In some embodiments, the oligonucleotide described herein comprises at least one chemically modified adenine. Exemplary chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6, N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2′-O-methyl-adenosine, N6, 2′-O-dimethyl-adenosine, N6-Methyl-2′-deoxyadenosine, N6, N6, 2′-O-trimethyl-adenosine, 1,2′-O-dimethyl-adenosine, 2′-O—ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the chemical modification described herein comprises modifying a guanine. In some embodiments, the oligonucleotide described herein comprises at least one chemically modified guanine. Exemplary chemically modified guanine can include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2, 7-dimethyl-guanosine, N2, N2, 7-dimethyl-guanosine, 8-oxo-guanosine, 7-methyl-8-oxo-guanosine, 1-meththio-guanosine, N2-methyl-6-thio-guanosine, N2,N2-dimethyl-6-thio-guanosine, a-thio-guanosine, 2′-O-methyl-guanosine, N2-methyl-2′-O—methyl-guanosine, N2,N2-dimethyl-2′-O-methyl-guanosine, 1-methyl-2′-O-methyl-guanosine, N2, 7-dimethyl-2′-O-methyl-guanosine, 2′-O-methyl-inosine, 1, 2′-O-dimethyl-inosine, 6—O-phenyl-2′-deoxyinosine, 2′-O-ribosylguanosine, 1-thio-guanosine, 6-O-methyguanosine, 06-Methyl-2′-deoxyguanosine, 2′-F-ara-guanosine, and 2′-F-guanosine.

In some embodiments, the chemical modification of the oligonucleotide can include introducing or substituting a nucleic acid analog or an unnatural nucleic acid into the oligonucleotide. In some embodiments, nucleic acid analog can be any one of the chemically modified nucleic acid described herein. all of which are expressly incorporated by reference in their entireties. The chemically modified nucleotide described herein can include a variant of guanosine, uridine, adenosine, thymidine, and cytosine, including any natively occurring or non-natively occurring guanosine, uridine, adenosine, thymidine or cytidine that has been altered chemically, for example by acetylation, methylation, hydroxylation. Exemplary chemically modified nucleotide can include 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O—methyl-2′-deoxycytidine, 2 ‘-O-methyl-2’-deoxyguanosine, 2, —O-methyl-2′-deoxyuridine, 2′-O—methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytosine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. In some embodiments, the chemically modified nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 6-methylguanosine-5′-triphosphate, pseudouridine-5′-triphosphate, puromycin-5′-triphosphate, or xanthosine-5′-triphosphate. In some embodiments, the chemically modified nucleic acid as described herein comprises at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-th io-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the chemically modified nucleic acid as described herein comprises at least one chemically modified nucleotide selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In other embodiments, the chemically modified nucleic acid as described herein comprises at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In certain embodiments, the chemically modified nucleic acid as described herein comprises at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

A modified base of a unnatural nucleic acid includes, but is not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, O-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and O-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C═C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine ([5,4-b][1,4]benzoxazin-2 (3H)-one), phenothiazine cytidine (1H-pyrimido[5,4-b][1,4]benzothiazin-2 (3H)-one), G-clamps, phenoxazine cytidine (e.g. 9-(2-aminoethoxy)-H-pyrimido[5,4-b][1,4]benzoxazin-2 (3H)-one), carbazole cytidine (2H-pyrimido[4,5-b]indol-2-one), pyridoindole cytidine (H-pyrido[3′,2′: 4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, or 2-amino-2′-deoxyadenosine.

In some embodiments, the at least one chemical modification comprises chemically modifying the 5′ or 3′ end. In some embodiments, the oligonucleotide comprises a chemical modification comprising 3′ nucleotides which can be stabilized against degradation, e.g., by incorporating one or more of the modified nucleotides described herein. In this embodiment, uridines can be replaced with modified uridines, e.g., 5-(2-amino) propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some embodiments, deaza nucleotides, e.g., 7-deaza-adenosine, can be incorporated into the oligonucleotide. In some embodiments, O- and N-alkylated nucleotides, e.g., N6-methyladenosine, can be incorporated into the oligonucleotide. In some embodiments, sugar-modified ribonucleotides can be incorporated, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN).

Chemical means for introducing the oligonucleotide into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An exemplary colloidal system for use as a delivery vehicle in vitro and in vivo is a liposome (e.g., an artificial membrane vesicle). Other methods of state-of-the-art targeted delivery of nucleic acids are available, such as delivery of oligonucleotide with targeted nanoparticles or other suitable sub-micron sized delivery system.

An exemplary delivery vehicle is a liposome. The use of lipid formulations is contemplated for the introduction of the oligonucleotide into a cell (in vitro, ex vivo or in vivo). In another aspect, the oligonucleotide can be associated with a lipid. The oligonucleotide associated with a lipid, in some embodiments, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, In some embodiments, they are present in a bilayer structure, as micelles, or with a “collapsed” structure. Alternately, they are simply be interspersed in a solution, possibly forming aggregates that are not uniform in size or shape. Lipids are fatty substances which are, In some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

“Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, in some embodiments, assume a micellar structure or merely exist as nonuniform aggregates of lipid molecules. Also contemplated are lipofectamine-nucleic acid complexes.

In some embodiments, the delivery method comprises lipofection, nucleofection, microinjection, biolistics, virosomes, liposomes, immunoliposomes, exosomes, polycation or lipid: cargo conjugates (or aggregates), naked polypeptide (e.g., recombinant polypeptides), naked DNA, artificial virions, and agent-enhanced uptake of polypeptide or DNA. In some embodiments, the delivery method comprises conjugating or encapsulating the compounds or the oligonucleotides described herein with at least one polymer such as natural polymer or synthetic materials. The polymer can be biocompatible or biodegradable. Non-limiting examples of suitable biocompatible, biodegradable synthetic polymers can include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, and poly(anhydrides). Such synthetic polymers can be homopolymers or copolymers (e.g., random, block, segmented, graft) of a plurality of different monomers, e.g., two or more of lactic acid, lactide, glycolic acid, glycolide, epsilon-caprolactone, trimethylene carbonate, p-dioxanone, etc. In an example, the scaffold can be comprised of a polymer comprising glycolic acid and lactic acid, such as those with a ratio of glycolic acid to lactic acid of 90/10 or 5/95. Non-limiting examples of naturally occurring biocompatible, biodegradable polymers can include glycoproteins, proteoglycans, polysaccharides, glycosamineoglycan (GAG) and fragment(s) derived from these components, elastin, laminins, decrorin, fibrinogen/fibrin, fibronectins, osteopontin, tenascins, hyaluronic acid, collagen, chondroitin sulfate, heparin, heparan sulfate, ORC, carboxymethyl cellulose, and chitin.

In some embodiments, the oligonucleotide described herein can be packaged and delivered to the cell via extracellular vesicles. The extracellular vesicles can be any membrane-bound particles. In some embodiments, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some embodiments, the extracellular vesicles can be exosomes, microvesicles, retrovirus-like particles, apoptotic bodies, apoptosomes, oncosomes, exophers, enveloped viruses, exomeres, or other very large extracellular vesicles.

In some embodiments, the oligonucleotide described herein is conjugated. In some embodiments, the oligonucleotide is conjugated to an aptamer, peptide, antibody, lipid, carbohydrate, or polymer. In some embodiments, the oligonucleotide is conjugated to an aptamer, peptide, antibody, lipid, carbohydrate, or polymer at the 5′ end of the oligonucleotide. In some embodiments, the oligonucleotide is conjugated to an aptamer peptide, antibody, lipid, carbohydrate, or polymer at the 3′ end of the oligonucleotide. In some embodiments, the oligonucleotide is conjugated to an aptamer, peptide, antibody, lipid, carbohydrate, or polymer at any nucleic acid residue of the oligonucleotide. In some embodiments, the aptamer, peptide, antibody, lipid, carbohydrate, or polymer conjugated to the oligonucleotide confers therapeutic effect. For example, the peptide, antibody, lipid, carbohydrate, or polymer conjugated to the oligonucleotide can be cytotoxic drug or drug for treating cancer. In some embodiments, the aptamer, peptide, antibody, lipid, carbohydrate, or polymer conjugated to the oligonucleotide increases the efficiency of the oligonucleotide binding to the endogenous nucleic acid. In some embodiments, the aptamer, peptide, antibody, lipid, carbohydrate, or polymer conjugated to the oligonucleotide confers targeting specificity of the oligonucleotide to specific types of cells (e.g., cancer cells, etc.). In some embodiments, the aptamer, peptide, antibody, lipid, carbohydrate, or polymer conjugated to the oligonucleotide confers stability of the oligonucleotide in vitro, ex vivo, or in vivo. For example, the oligonucleotide can be conjugated with polyethylene glycol (PEG) or endosomolytic agent to decrease immunogenicity or degradation. In some embodiments, the aptamer, peptide, antibody, lipid, carbohydrate, or polymer conjugated to the oligonucleotide to facilitate the oligonucleotide for entering cell. In some embodiments, the aptamer, peptide, antibody, lipid, carbohydrate, or polymer conjugated to the oligonucleotide to facilitate and release to the oligonucleotide in the cell. In some embodiments, the aptamer, peptide, antibody, lipid, carbohydrate, or polymer conjugated to the oligonucleotide comprises at least one targeting moiety for targeting the cell. Non-limiting examples of the targeting moiety comprises a signaling peptide, a chemokine, a chemokine receptor, an adhesion molecule, an antigen, or an antibody.

In embodiments, the antisense oligonucleotide described herein is conjugated to an aptamer that targets pancreatic cells, as described for example in PCT/US2023/026980, which is hereby incorporated by reference in its entirety.

The linker for conjugating the oligonucleotide to the aptamer, peptide, antibody, lipid, or polymer can be any linker that connects biomolecules. In some embodiments, a linker described herein is a cleavable linker or a non-cleavable linker. In some instances, the linker is a cleavable linker. In other instances, the linker is a non-cleavable linker. In some embodiments, the linker is a non-polymeric linker. A non-polymeric linker refers to a linker that does not contain a repeating unit of monomers generated by a polymerization process. In some embodiments, the linker comprises a peptide moiety. In some instances, the peptide moiety comprises at least 2, 3, 4, 5, or 6 more amino acid residues. In some embodiments, the linker comprises a benzoic acid group, or its derivatives thereof. In some embodiments, the linker can comprise nucleic acid linker such as DNA linker. In such case, the aptamer, peptide, antibody, lipid, or polymer can be conjugated on one end of the nucleic acid linker or intercalated into the nucleic acid base pairing of the nucleic acid linker. In some embodiments, the linker can be a peptide linker. The peptide linker can be flexible (e.g., poly-glycine linker) or rigid (e.g., EAAAK repeat linker). In some embodiments, the peptide linker can be cleaved (e.g., a disulfide bond). In some embodiments, the linker comprises polymers such PEG, polylactic acid (PLA), or polyacrylic acid (PAA).

In embodiments, the melting temperature of the antisense oligonucleotide hybridized to its target sequence is at least about 35° C. The Tₘ of an oligonucleotide is the temperature at which 50% of the oligonucleotide is duplexed with its perfect complement and 50% is free in solution. The Tm can be determined experimentally by measuring the absorbance change of the oligonucleotide with its complement as a function of temperature. The Tₘ can also be estimated using known publicly available Tₘ calculators. In some embodiments, the Tₘ of the oligonucleotide hybridized to its target sequence is at least about 40° C., or at least about 45° C., or at least about 50° C. In some embodiments, the Tₘ of the oligonucleotide hybridized to its target sequence is from about 35° C. to about 60° C. In some embodiments, the Tₘ of the oligonucleotide hybridized to its target sequence is from about 40° C. to about 60° C., or from about 50° C. to about 60° C.

In some embodiments, the antisense oligonucleotide further comprises a cell penetrating moiety, which in some embodiments is conjugated directly or indirectly at or to the 3′ end of the oligonucleotide, and optionally though a linker (e.g., a polyethylene glycol linker or alkyl linker). In some embodiments, the compound further comprises a sterol conjugate (e.g., cholesterol conjugate) or fatty acid conjugate such as a palmitoyl or stearyl lipid conjugate, which is optionally conjugated to the 3′ end of the antisense oligonucleotide. These moieties can enhance cell penetration. See U.S. Pat. No. 9,012,225, which is hereby incorporated by reference in its entirety.

In some embodiments, the compound does not comprise any encapsulation or transfection reagent.

In some embodiments, the antisense oligonucleotide is encapsulated in a particle. In various embodiments, the particle is a liposome, polymeric nanoparticle, or lipid nanoparticle. Exemplary polymeric nanoparticles can be formed of PLA, PLGA, or PEG copolymers thereof. In some embodiments, the particle comprises poly(β amino ester) polymers. In various embodiments, the LNPs comprise a cationic or ionizable lipid, a neutral lipid, a cholesterol or cholesterol moiety, and a PEGylated lipid.

In some embodiments, the lipid nanoparticle (or LNP) comprises a structural lipid. Exemplary structural lipids can be selected from one or more of cholesterol, fecosterol, sitosterol, ergosterol, campesterol, stigmasterol, brassicasterol, tomatidine, ursolic acid, and tocopherols (e.g., alpha tocopherol). In some embodiments, the structural lipid is cholesterol.

In some embodiments, the LNP comprises one or more phospholipids. Exemplary phospholipids are selected from cardiolipins, sterol modified lipids (modified with a cholesterol moiety attached at the sn-2 carbon of the glycerol backbone), mixed-acyl glycerophospholipids, and symmetrical acyl glycerophospholipids. Head groups for acyl glycerophospholipids include, for example, phosphatidic acid, lysophosphatidic acid, phosphatidylcholine, phosphatidylethanolamine, phosphatidylglycerol, phosphoinositides, and phosphatidylserine. Exemplary phospholipids are selected from 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanol amine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

In some embodiments, the lipid nanoparticle composition further comprises one or more PEG lipids. A PEG lipid is a lipid modified with polyethylene glycol. Exemplary PEG lipids are selected from one or more of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol. A PEG lipid may be selected from PEG-c-DOMG, PEG-DMG, PEG-DLPE, PEG-DMPE, PEG-DPPC, PEG-Cholesterol, PEG tocopherol, or a PEG-DSPE lipid.

Lipid particle formulations that find use with embodiments of the present disclosure include those described in U.S. Pat. Nos. 9,738,593; 10,221,127; 10,166,298, which are hereby incorporated by reference in their entirety. In some embodiments, the liposomes or nanoparticles further comprise a targeting moiety as described.

In other embodiments, the compound is formulated for parenteral administration. In some embodiments, the compound for parenteral administration comprises encapsulation in a particle as described.

Disclosed herein, in some embodiments, are methods of modulating KRAS-mediated signaling pathway in a cancer cell by treating or contacting the cancer cell with a composition comprising antisense oligonucleotide, composition, or pharmaceutical composition described herein, thereby reducing expression of KRAS or mutated KRAS protein or mRNA in the cancer cell. In some embodiments, mutated KRAS protein comprising a G12C mutation, a G12V mutation, a G12A mutation, or a G12D mutation.

Also disclosed herein, in some embodiments, are methods of treating a subject in need thereof by administrating a therapeutic effective amount of the oligonucleotide, composition, or pharmaceutical composition described herein to the subject. In some embodiments, the method treats the subject by modulating gene expression associated with a signaling pathway or an activity of a signaling pathway expression in the subject. In some embodiments, the method comprises decreasing gene expression by contacting a nucleic acid (e.g., endogenous mRNA) or a cell (e.g., a cancer cell) comprising the nucleic acid with the oligonucleotide described herein. In some embodiments, the method comprises decreasing KRAS, mutated KRAS, or a combination of KRAS and mutated KRAS in the subject or in the cancer cell by contacting mRNA of KRAS or mutated KRAS with the oligonucleotide described herein, where the binding of the oligonucleotide to the mRNA recruits endogenous nuclease for degradation of the mRNA. In some embodiments, the method comprises decreasing expression of signaling pathway such as KRAS-mediated signaling pathway. In some embodiments, the method comprises decreasing expression of a gene in or the activity of the KRAS-RAF-MEK-ERK signaling pathway, the PI3K signaling pathway, the MAPK signaling pathway, or the Ral-GEF signaling pathway.

In some embodiments, the oligonucleotide, composition, or pharmaceutical composition can be administered to the subject alone (e.g., standalone treatment). In some embodiments, the oligonucleotide, composition, or pharmaceutical composition is administered in combination with an additional agent. In some embodiments, the additional agent as used herein is administered alone. The oligonucleotide, composition, or pharmaceutical composition and the additional agent can be administered together or sequentially. Non-limiting examples of the additional agent comprise N-(2-(4-(4-bis(2-chloroethyl) aminophenyl) butyryl) aminoethyl)-5-(4-amidinophenyl)-2-furanecarboxamide hydrochloride; Allyl isothiocyanate; Benzyl isothiocyanate; Phenethyl isothiocyanate; Belinostat; Berberin; Casticin; Chrysin; Bufalin; Fisetin; Fucoidan; Galic acid; Gemcitabine; Guizhi Fuling Decoction; JOTO1007; Quercetin; Rasfonin; 2,3,7,8-tetrachlorodibenzodioxin; Triptolide; 4-Hydroxybutenolide; or a combination thereof. The combination therapies can be administered within the same day, or can be administered one or more days, weeks, months, or years apart.

In some embodiments, the oligonucleotide, composition, or pharmaceutical composition is a first-line treatment for the disease or condition. In some embodiments, the oligonucleotide, composition, or pharmaceutical composition is a second-line, third-line, or fourth-line treatment. In general, method disclosed herein comprises administering the oligonucleotide, composition, or pharmaceutical composition by oral administration. However, in some instances, method comprises administering the oligonucleotide, composition, or pharmaceutical composition by intraperitoneal injection. In some instances, the method comprises administering the pharmaceutical composition in the form of an anal suppository. In some instances, the method comprises administering the oligonucleotide, composition, or pharmaceutical composition by intravenous (“i.v.”) administration. It is conceivable that one can also administer the oligonucleotide, composition, or pharmaceutical composition disclosed herein by other routes, such as subcutaneous injection, intramuscular injection, intradermal injection, transdermal injection percutaneous administration, intranasal administration, intralymphatic injection, rectal administration intragastric administration, or any other suitable parenteral administration. In some embodiments, routes for local delivery closer to site of injury or inflammation are preferred over systemic routes. Routes, dosage, time points, and duration of administrating therapeutics can be adjusted. In some embodiments, administration of therapeutics is prior to, or after, onset of either, or both, acute and chronic symptoms of the disease or condition.

Suitable dose and dosage administrated to a subject is determined by factors including, but no limited to, the particular the oligonucleotide, composition, or pharmaceutical composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject being treated.

The effective dosage ranges can be adjusted based on subject's response to the treatment. Some routes of administration will require higher concentrations of effective amount of therapeutics than other routes.

In some embodiments, the administration of the oligonucleotide, composition, or pharmaceutical composition described herein inhibits growth of the tumor by at least 10%, 15%, 20%, 30%, 40%, 50%, or more. In some embodiments, the administration of the oligonucleotide, composition, or pharmaceutical composition described herein at a dose that inhibits growth of the tumor by at least 10%, 15%, 20%, 30%, 40%, 50%, or more. In some embodiments, the administration of the oligonucleotide, composition, or pharmaceutical composition described herein at a schedule that inhibits growth of the tumor by at least 10%, 15%, 20%, 30%, 40%, 50%, or more. In some embodiments, the administration of the oligonucleotide, composition, or pharmaceutical composition described herein at a dose and a schedule that inhibits growth of the tumor by at least 10%, 15%, 20%, 30%, 40%, 50%, or more.

In some embodiments, the administration of the oligonucleotide, composition, or pharmaceutical composition described herein to the subject in a dose that is sufficient to inhibit growth of the tumor. In some embodiments, the administration of the oligonucleotide, composition, or pharmaceutical composition described herein to the subject in a schedule that is sufficient to inhibit growth of the tumor. In some embodiments, the administration of the oligonucleotide, composition, or pharmaceutical composition described herein to the subject in a dose and a schedule that are sufficient to inhibit growth of the tumor.

In some embodiments, the disease or condition described herein is a cancer. In some embodiments, the cancer is associated with KRAS. In some embodiments, the cancer is associated with mutated KRAS. In some embodiments, the cancer is associated with KRAS. In some embodiments, the cancer is associated with an abnormality of KRAS-mediated signaling pathway. In some embodiments, the cancer is a lung cancer, a pancreatic cancer, or a colon cancer. Other non-limiting examples of the cancer can include Acute Lymphoblastic Leukemia, Acute Lymphocytic Leukemia (ALL), Acute Myeloid Leukemia (AML), Adenoid Cystic Carcinoma, Adrenal Gland Cancer, Adrenocortical Carcinoma, Adult Leukemia, AIDS-Related Lymphoma, Amyloidosis, Anal Cancer, Astrocytomas, Ataxia Telangiectasia, Atypical Mole Syndrome, Atypical Teratoid/Rhabdoid Tumor, Basal Cell Carcinoma, Bile Duct Cancer, Birt Hogg Dube Syndrome, Bladder Cancer, Bone Cancer, Brain Tumor, Breast Cancer, Bronchial Tumors, Burkitt Lymphoma, Carcinoid Tumor (Gastrointestinal), Carcinoma of Unknown Primary, Cardiac (Heart) Tumors, Cervical Cancer, Cholangiocarcinoma, Chordoma, Chronic Lymphocytic Leukemia (CLL), Chronic Myelogenous Leukemia, Chronic Myeloid Leukemia, Chronic Myeloproliferative Neoplasms, Colorectal Cancer, Craniopharyngioma, Cutaneous T-Cell Lymphoma, Ductal Carcinoma, Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer, Fallopian Tube Cancer, Fibrous Histiocytoma of Bone, Malignant, and Osteosarcoma, Gallbladder Cancer, Gastric Cancer, Gastrointestinal Carcinoid Tumor, Gastrontestinal Stromal Tumor (GIST), Germ Cell Tumors, Gestational Trophoblastic Disease, Hairy Cell Leukemia, Head and Neck Cancer, Hepatocellular Cancer, HER2-Positive Breast Cancer, Histiocytosis, Langerhans Cell, Hodgkin's Lymphoma, Hypopharyngeal Cancer, Intraocular Melanoma, Islet Cell Tumor, Juvenile Polyposis Syndrome, Kaposi Sarcoma, Kidney Cancer, Langerhans Cell Histiocytosis, Laryngeal Cancer, Leukemia, Lip and Oral Cavity Cancer, Liver Cancer, Lobular Carcinoma, Lung Cancer (Non-Small Cell and Small Cell), Lymphoma, Malignant Fibrous Histiocytoma of Bone and Osteosarcoma, Malignant Glioma, Melanoma, Intraocular Melanoma, Meningioma, Merkel Cell Carcinoma, Mesothelioma, Malignant, Metastatic Cancer, Metastatic Squamous Neck Cancer with Occult Primary, Midline Tract Carcinoma, Multiple Endocrine Neoplasia Syndromes, Multiple Myeloma, Plasma Cell Neoplasms, Mycosis Fungoides, Myelodysplastic Syndrome (MDS), Myeloproliferative Neoplasms, Chronic, Nasal Cavity and Paranasal Sinus Cancer, Nasopharyngeal Cancer, Neuroblastoma, Neuroendocrine Tumor, Non-Hodgkin Lymphoma, Oral Cancer, Lip and Oral Cavity Cancer and Oropharyngeal Cancer, Oropharyngeal Cancer, Osteosarcoma, Ovarian Cancer, Ovarian Germ Cell Tumors, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Peritoneal Cancer, Peutz-Jeghers Syndrome, Pharyngeal Cancer, Pheochromocytoma, Pituitary Tumor, Plasma Cell Neoplasm/Multiple Myeloma, Pleuropulmonary Blastoma, Polycythemia Vera, Pregnancy and Breast Cancer, Primary Central Nervous System (CNS) Lymphoma, Primary Peritoneal Cancer, Prostate Cancer, Rectal Cancer, Recurrent Cancer, Renal Cell Carcinoma, Retinoblastoma, Rhabdomyosarcoma, Salivary Gland Cancer, Sarcoma, Sézary Syndrome, Skin Cancer, Small Intestine Cancer, Soft Tissue Sarcoma, Solid tumor, Squamous Cell Carcinoma of the Skin, Squamous Neck Cancer with Occult Primary, Metastatic, Stomach Cancer, T-Cell Lymphoma, Testicular Cancer, Throat Cancer, Thymoma, Thymic Carcinoma, Thyroid Cancer, Transitional Cell Cancer of the Renal Pelvis and Ureter, Unusual Cancers of Childhood, Ureter and Renal Pelvis, Transitional Cell Cancer, Urethral Cancer, Uterine (Endometrial) Cancer, Uterine Sarcoma, Vaginal Cancer, Vascular Tumors, Vulvar Cancer, Wilms Tumor, or a combination thereof.

In some embodiments, the cancer is pancreatic cancer.

Described herein, in some embodiments, is a pharmaceutical composition comprising the oligonucleotide or the compound described herein. Pharmaceutical composition, as used herein, refers to a mixture of a pharmaceutical composition, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compounds include two or more pharmaceutical composition as discussed herein. In practicing the methods of treatment or use provided herein, therapeutically effective amounts of pharmaceutical compositions described herein are administered in a pharmaceutical composition to a mammal having a disease, disorder, or condition to be treated, e.g., an inflammatory disease, fibrostenotic disease, and/or fibrotic disease. In some embodiments, the mammal is a human. A therapeutically effective amount can vary widely depending on the severity of the disease, the age and relative health of the subject, the potency of the pharmaceutical composition used and other factors. The pharmaceutical compositions can be used singly or in combination with one or more pharmaceutical compositions as components of mixtures. The pharmaceutical commotions described herein comprise the oligonucleotide, the compounds, the cells contacted with the oligonucleotide or contacted with the compound comprising the oligonucleotide, or a combination thereof.

The pharmaceutical formulations described herein are administered to a subject by appropriate administration routes, including but not limited to, intravenous, intratumoral, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a pharmaceutical composition are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a pharmaceutical composition as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, pharmaceutical compositions exist in unsolvated form or in solvated forms with pharmaceutically acceptable solvents such as water, ethanol, and the like. The solvated forms of the pharmaceutical compositions are also considered to be disclosed herein.

In some embodiments, a pharmaceutical composition exists as an enantiomer, diastereomer, or other steroisomeric form. The agents disclosed herein include all enantiomeric, diastereomeric, and epimeric forms as well as mixtures thereof.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some embodiments, context may dictate a particular meaning.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to +10% of a stated number or value.

The terms “increased”, “increasing”, or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

### EXAMPLES

The following illustrative examples are representative of embodiments of the present disclosure.

### Example 1. Exemplary KRAS Antisense Oligonucleotide and Knockdown of KRAS mRNA

TABLE 1
Exemplary KRAS antisense oligonucleotide

| | Sequence | |
|---|---|---|
| | Identifier for | |
| Oligo | Nucleobase | |
| Number | Sequence | Antisense Oligonucleotide |
| STN-001 | SEQ ID NO: 7 | +T*+A*+C*G*C*C*A*C*A*A*G*+C*+T*+C |
| STN-002 | SEQ ID NO: 8 | +A*+C*+G*C*C*A*C*A*A*G*C*+T*+C |
| STN-003 | SEQ ID NO: 8 | +A*+C*G*C*C*A*C*A*A*G*+C*+T*+C |
| STN-004 | SEQ ID NO: 14 | +C*+T*+A*C*G*C*C*A*C*A*A*+G*+C*+T |
| STN-005 | SEQ ID NO: 15 | +C*+T*+A*C*G*C*C*A*C*A*A*G*+C*+T*+C |
| STN-006 | SEQ ID NO: 9 | +A*+C*+G*C*C*A*C*A*A*G*/15HydMe-dC/*+T*+C*+C |
| STN-007 | SEQ ID NO: 9 | +A*+C*+G*C*/12MOErC/*A*C*A*A*G*C*+T*+C*+C |
| STN-008 | SEQ ID NO: 9 | +A*+C*+G*C*/12MOErC/*A*C*A*A*G*/i5HydMe-dC/*+T*+C*+C |
| STN-009 | SEQ ID NO: 9 | +A*+C*+G*C*C*A*C*A*A*/12MOErG/*C*+T*+C*+C |
| STN-010 | SEQ ID NO: 9 | +A*+C*+G*C*C*A*C*A*A*/12MOErG/*/i5HydMe-dC/*+T*+C*+C |
| STN-011 | SEQ ID NO: 7 | TACGCCACAAGCTC |
| STN-012 | SEQ ID NO: 8 | ACGCCACAAGCTC |
| STN-013 | SEQ ID NO: 14 | CTACGCCACAAGCT |
| STN-014 | SEQ ID NO: 15 | CTACGCCACAAGCTC |
| STN-015 | SEQ ID NO: 9 | ACGCCACAAGCTCC |

“+” indicates LNA, and may be optionally replaced with 2′-MOE or 2′-OMe, or other bridged nucleotide such as cEt;
“*” indicates a phosphorothioate internucleotide linkage;
“i2MOEr” indicates an internucleotide 2-MethoxyEthoxy modified nucleotide;
“i5HydMe” indicates an internucleotide 5-hydroxymethyl modified nucleotide.

TABLE 2
Additional antisense oligonucleotide

| | Sequence | |
|---|---|---|
| | Identifier for | |
| Oligo | Nucleobase | |
| Number | Sequence | Antisense Oligonucleotide |
| STN-016 | SEQ ID NO: 16 | +T*+A*+C*G*C*C*A*C*A*A*G*C*T*+C*+C*+A |
| STN-017 | SEQ ID NO: 17 | +A*+C*+G*C*C*A*C*A*A*G*C*T*+C*+C*+A |
| STN-018 | SEQ ID NO: 9 | +A*+C*+G*C*C*A*C*A*A*G*C*+T*+C*+C |
| STN-019 | SEQ ID NO: 9 | +A*+/iMeC/*+G*/iMeC/*/iMeC/*A*/iMeC/ |
| | | *A*A*G*/iMeC/*+T*+/iMeC/*+/3MeC |
| STN-020 | SEQ ID NO: 18 | +C*+G*+C*C*A*C*A*A*G*C*T*+C*+C*+A |
| STN-021 | SEQ ID NO: 19 | +C*+G*+C*C*A*C*A*A*G*C*+T*+C*+C |
| STN-022 | SEQ ID NO: 20 | +A*+C*+G*T*C*T*A*T*A*C*A*+C*+C*+A |
| STN-023 | SEQ ID NO: 16 | TACGCCACAAGCTCCA |
| STN-024 | SEQ ID NO: 17 | ACGCCACAAGCTCCA |
| STN-025 | SEQ ID NO: 18 | CGCCACAAGCTCCA |
| STN-026 | SEQ ID NO: 9 | ACGCCACAAGCTCC |
| STN-027 | SEQ ID NO: 18 | CGCCACAAGCTCCA |
| STN-028 | SEQ ID NO: 19 | CGCCACAAGCTCC |
| STN-029 | SEQ ID NO: 20 | ACGTCTATACACCA |
| STN-030 | SEQ ID NO: 21 | +C*+A*+C*G*T*C*T*A*T*A*C*A*C*+C*+A*+C |
| STN-031 | SEQ ID NO: 21 | CACGTCTATACACCAC |

“+” indicates LNA, and may be optionally replaced with 2′-MOE or 2′-OMe, or other bridged nucleotide such as cEt;
“*” indicates a phosphorothioate internucleotide linkage;
“iMe” indicates an internucleotide 5-methyl modified nucleotide.
“3Me” indicates a 3′ 5-methyl modified nucleotide.

Knockdown of KRAS mRNA

### Cell Culture Condition and In Vitro Transfection

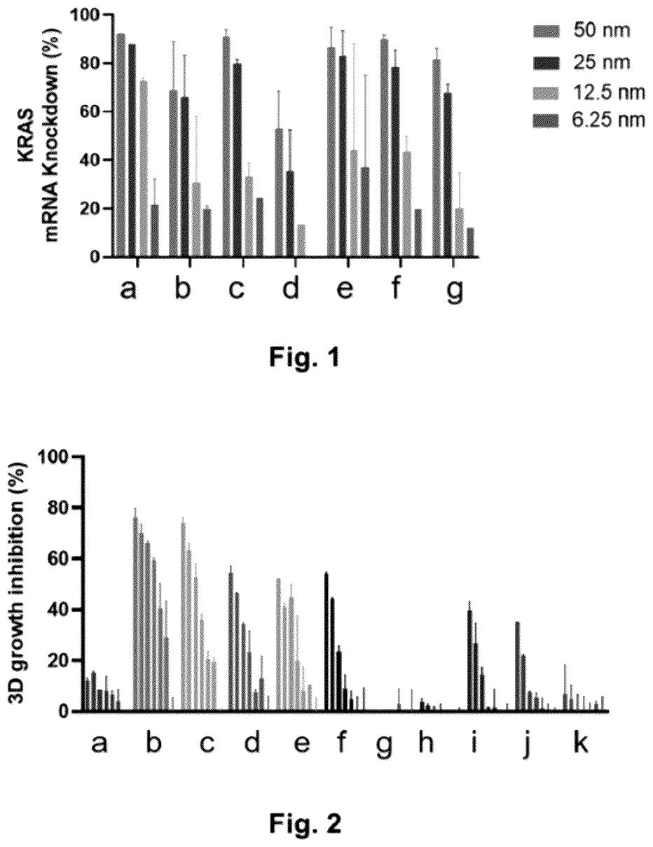

FIG. 1 depicts mutated KRAS mRNA knockdown by KRAS G12C-specific antisense oligonucleotides (ASOs). The y-axis shows the percentage of KRAS mRNA knockdown, relative to KRAS mRNA in the cells treated with STN-030, a non-KRAS-targeting ASO control. The x-axis, from left to right, shows the ASO used: a-STN-016; b-STN-017; c-STN-018; d-STN-019; e-STN-001; f-STN-002; g-STN-003. To avoid TCC nucleotides at the 3′end, STN-001 has a base shift relative to STN-018; STN-002 is a 13-mer as wing-gap-wing configuration of 3-8-2; STN-003 is a 13-mer as wing-gap-wing configuration of 2-8-3. Mia PaCa-2 (ATCC) cells carrying KRAS G12C mutation were plated at a density of 20,000 cells per well in a clear and flat bottom 96-well plates in RPMI1640 with 10% FBS without antibiotics. The cells were transfected with 50 nM, 25 nM, 12.5 nM, or 6.25 nM of antisense oligonucleotide complexed with RNAiMAX (Thermo Fisher). Transfection mixture was removed and replenished with RPMI1640+10% FBS, penicillin/streptomycin after overnight incubation at 37° C., and incubated for an additional 48 hours. The mRNA quantitation was performed using QuantiGene (Thermo Fisher) according to manufacturer's instructions. To normalize inter- and intra-experimental variation, STN-018 was selected as the benchmark ASO and included in all plates to normalize the mRNA knockdown.

### Example 2. Antisense Oligonucleotide Mediated Growth Inhibition of Cancer Cells

FIG. 2 and Table 3 below depict 3D Growth Inhibition of G12C Specific ASOs in Mia PaCa-2 cells. The y-axis shows the percentage of growth inhibition, relative to STN-030, a non-KRAS-targeting ASO control. The x-axis, from left to right, shows the ASO used: a-STN-022; b-STN-016; c-STN-017; d-STN-025; e-STN-018; f-STN-020; g-STN-021; h-STN-019; i-STN-001; j-STN-002; k-STN-003.

Cells were plated in clear 384 well plates (S-Bio, #MS-9384UZ) at 800 cells per well in RPMI1640 with 10% FBS for overnight and treated with 5 μM, 2.5 μM, 1.25 μM, 0.625 μM, 0.313 μM, 0.156 μM, or 0.078 UM ASO. After 5 days, cell proliferation was determined by measuring total ATP content using the Cell Titer Glo reagent (Promega, G7570) according to manufacturer's instructions. In FIG. 2, for each group of cells treated with a specific ASO, each individual bar, from left to right, depicts highest to lowest concentrations of ASO used (2-fold dilution from left to right). The ASOs used in this experiment exhibit significant growth inhibition activity against cancer cell lines.

TABLE 3
3D Growth Inhibition of G12C specific ASOs MIA PaCa-2 cell line

| | | | 3D Proliferation | |
|---|---|---|---|---|
| | Antisense Oligonucleotide | | Assay | |
| Oligo | (Sequence identifier for | | Inhibition (%) | |
| Number | nucleobase sequence) | Target | 5 μM | 1.25 μM |
| STN-001 | +T*+A*+C*G*C*C*A*C*A*A*G*+C*+T*+C | hKRAS | 61.7 | 35.2 |
| | (SEQ ID NO: 7) | G12C | | |
| STN-002 | +A*+C*+G*C*C*A*C*A*A*G*C*+T*+C | hKRAS | 62.0 | 40.4 |
| | (SEQ ID NO: 8) | G12C | | |
| STN-003 | +A*+C*G*C*C*A*C*A*A*G*+C*+T*+C | hKRAS | 53.7 | 32.0 |
| | (SEQ ID NO: 8) | G12C | | |
| STN-004 | +C*+T*+A*C*G*C*C*A*C*A*A*+G*+C*+T | hKRAS | 65.8 | 42.3 |
| | (SEQ ID NO: 14) | G12C | | |
| STN-005 | +C*+T*+A*C*G*C*C*A*C*A*A*G*+C*+T*+C | hKRAS | 61.6 | 33.5 |
| | (SEQ ID NO: 15) | G12C | | |

“+” indicates LNA, and may be optionally replaced with 2′-MOE or 2′-OMe, or other bridged nucleotide such as cEt; indicates a phosphorothioate internucleotide linkage.

### Example 3. Mutated KRAS mRNA Knockdown and KRAS Pathway Modulation

### Cell Line

TABLE 5
Different cell lines were used to
evaluate mutant specific KRAS ASOs:

| Name | Accession | Genotype |
|---|---|---|
| MIA PaCa-2 | CVCL_0428 | KRAS G12C |
| PANC-1 | CVCL_0480 | KRAS G12D |
| NCI-H2009 | CVCL_1514 | KRAS G12A |
| TCC-PAN2 | CVCL_3178 | KRAS G12R |
| CAPAN-1 | CVCL_0237 | KRAS G12V |
| LCLC-97TM1 | CVCL_1376 | KRAS G12V |
| A-375 | CVCL_0132 | KRAS WT |

mRNA Knockdown

The ability of ASOs to knockdown desired mRNA was assessed as follows. Cells were plated in clear, flat bottom 96 well plate at 20,000 cells per well in growth medium with 10-20% FBS with Pen/Strep 100 I.U/mL. ASOs were added to cells gymnotically at various concentrations. Treated cells were incubated for 96 hours at 37 C. Pan KRAS ASO (STN-100019) was used as positive control. The mRNA quantitation was performed using QuantiGene from Thermo Fisher according to its instructions. The results are shown in Table 6-11 below.

TABLE 6
KRAS mRNA Knockdown G12V Modified Specific Sequences in Capan-1 and
LCLC-97TM1 Cell Lines

| | | | Sequence | KRAS mRNA KD | |
|---|---|---|---|---|---|
| | | | Identifier | (%) | |
| | | | for | | LCLC97- |
| Oligo | | | Nucleobase | Capan-1 | TM1 |
| Number | Target | Antisense Oligonucleotide | Sequence | 5 μM | 5 μM |
| STN- | Pan | +C*+C*+T*C*A*T*T*G*C*A*C*T*G*+T*+A* | SEQ ID | 81.15 | 97.6 |
| 100019 | hKRAS | +C | NO: 1 | | |
| STN- | hKRAS | +T*+A*+C*G*C*C*A*A*C*A*G*+C*+T*+C | SEQ ID | 79.73 | 80.14 |
| 100080 | G12V | | NO: 2 | | |
| STN- | hKRAS | +T*+A*/iMe-dC/*G*/iMe-dC/*/iMe-dC/ | SEQ ID | 24.04 | n/a |
| 100940 | G12V | *A*A*/iMe-dC/*A*G*/iMe-dC/*+T*+C | NO: 2 | | |
| STN- | hKRAS | +T*+A*/iMe-dC/*G*C*C*A*A*C*A*G*/ | SEQ ID | 26.25 | n/a |
| 100941 | G12V | iMe-dC/*+T*+C | NO: 2 | | |
| STN- | hKRAS | +T*+A*C*G*C*C*A*A*C*A*G*C*+T*+C | SEQ ID | 38.52 | n/a |
| 100942 | G12V | | NO: 2 | | |
| STN- | hKRAS | +T*+A*+C*G*C*C*A*A*C*A*G*C*+T*+C | SEQ ID | 35.92 | n/a |
| 100943 | G12V | | NO: 2 | | |
| STN- | hKRAS | +T*+A*C*G*C*C*A*A*C*A*G*+C*+T*+C | SEQ ID | 68.92 | 89.88 |
| 100944 | G12V | | NO: 2 | | |
| STN- | hKRAS | +C*+T*+A*C*G*C*C*A*A*C*A*+G*+C*+T | SEQ ID | 63.43 | 43.49 |
| 100987 | G12V | | NO: 3 | | |
| STN- | hKRAS | +A*/iMe-dC/+G*C*C*A*A*C*A*G*+C*+ | SEQ ID | 33.62 | n/a |
| 100988 | G12V | T*+C | NO: 4 | | |
| STN- | hKRAS | +A*+C*+G*C*C*A*A*C*A*G*C*+T*+C*/ | SEQ ID | 38.96 | 74.2 |
| 100989 | G12V | 3Me-dC | NO: 5 | | |
| STN- | hKRAS | +A*+C*+G*C*C*A*A*C*A*G*C*+T*/iMe- | SEQ ID | 31.25 | 81.45 |
| 100990 | G12V | dC/+C | NO: 5 | | |
| STN- | hKRAS | +A*/iMe-dC/*G*C*C*A*A*C*A*G*C*+ | SEQ ID | 6.16 | n/a |
| 100991 | G12V | T*+C | NO: 4 | | |
| STN- | hKRAS | +A*+C*G*C*C*A*A*C*A*G*/iMe-dC/ | SEQ ID | 33.86 | 75.62 |
| 100992 | G12V | *+T*+C | NO: 4 | | |
| STN- | hKRAS | +A*+C*G*C*C*A*A*C*A*G*+C*+T*/ | SEQ ID | 67.73 | 83.56 |
| 100993 | G12V | 3Me-dC | NO: 4 | | |
| STN- | hKRAS | +T*+A*+C*G*C*C*A*A*C*A*G+C*+T*/ | SEQ ID | 65.62 | 53.95 |
| 100996 | G12V | 3Me-dC | NO: 2 | | |
| STN- | hKRAS | +A*/iMe- | SEQ ID | 40.17 | 30.12 |
| 101007 | G12V | dC/*+G*C*C*A*T*C*A*G*C*T*+C*/ | NO: 6 | | |
| | | iMe-dC/*+A | | | |

“+” indicates LNA, and may be optionally replaced with 2′-MOE or 2′-OMe, or other bridged nucleotide such as cEt;
“*” indicates a phosphorothioate internucleotide linkage;
“iMe” indicates an internucleotide 5-methyl modified nucleotide.
“3Me” indicates a 3′ 5-methyl modified nucleotide.

TABLE 7
KRAS mRNA Knockdown G12C Modified Specific Sequences
in Mia PaCa-2 Cell Line

| | | | Sequence | KRAS mRNA |
|---|---|---|---|---|
| | | | Identifier | KD (%) |
| | | | for | MIA |
| Oligo | | | Nucleobase | PaCa-2 |
| Number | Target | Antisense Oligonucleotide | Sequence | 5 μM |
| STN- | Pan | +C*+C*+T*C*A*T*T*G*C*A*C* | SEQ ID NO: 1 | 90.2 |
| 100019 | hKRAS | T*G*+T*+A*+C | | |
| STN- | hKRAS | +T*+A*+C*G*C*C*A*C*A*A*G*+ | SEQ ID NO: 7 | 71.4 |
| 100840 | G12C | C*+T*+C | | |
| STN- | hKRAS | +A*+C*+G*C*C*A*C*A*A*G*C*+ | SEQ ID NO: 8 | 67.3 |
| 100841 | G12C | T*+C | | |
| STN- | hKRAS | +A*+C*G*C*C*A*C*A*A*G*+C*+ | SEQ ID NO: 8 | 66.2 |
| 100842 | G12C | T*+C | | |
| STN- | hKRAS | +A*+C*+G*C*C*A*C*A*A*G*/ | SEQ ID NO: 9 | 61.7 |
| 100843 | G12C | i5HydMe-dC/*+T*+C*+C | | |
| STN- | hKRAS | +A*+C*+G*C*/12MOErC/*A*C* | SEQ ID NO: 9 | 74.5 |
| 100844 | 12C | A*A*G*C*+T*+C*+C | | |
| STN- | hKRAS | +A*+C*+G*C*/12MOErC/*A*C*A* | SEQ ID NO: 9 | 71.5 |
| 100845 | G12C | A*G*/i5HydMe-dC/*+T*+C*+C | | |
| STN- | hKRAS | +A*+C*+G*C*C*A*C*A*A*/ | SEQ ID NO: 9 | 67.9 |
| 100846 | G12C | 12MOErG/*C*+T*+C*+C | | |
| STN- | hKRAS | +T*+A*+/iMe-dC/*G*C*C*A*C* | SEQ ID NO: 7 | 66.4 |
| 100949 | 12C | A*A*G*+C*+T*+C | | |
| STN- | hKRAS | +T*+A*+/iMe-dC/*G*C*C*A*C* | SEQ ID NO: 7 | 68.1 |
| 100950 | G12C | A*A*G*C*+T*+C | | |

“+” indicates LNA, and may be optionally replaced with 2′-MOE or 2′-OMe, or other bridged nucleotide such as cEt;
“*” indicates a phosphorothioate internucleotide linkage;
“i2MOEr” indicates an internucleotide 2-MethoxyEthoxy modified nucleotide.
“i5HydMe” indicates an internucleotide 5-hydroxymethyl modified nucleotide.
“iMe” indicates an internucleotide 5-methyl modified nucleotide.

TABLE 8
KRAS mRNA Knockdown G12D Modified Specific Sequences in PANC-1 Cell Line

| | | | Sequence | KRAS mRNA |
|---|---|---|---|---|
| | | | Identifier for | KD (%) |
| | | | Nucleobase | PANC-1 |
| Number | Target | Antisense Oligonucleotide | Sequence | 5 μM |
| STN- | Pan | +C*+C*+T*C*A*T*T*G*C*A*C*T*G*+T*+A*+C | SEQ ID NO: 1 | 95.00 |
| 100019 | hKRAS | | | |
| STN- | hKRAS | +T*+A*+C*G*C*C*A*T*C*A*G*C*+T*+C*+C | SEQ ID NO: 10 | 55.40 |
| 100903 | G12D | | | |
| STN- | hKRAS | +A*+C*+G*C*C*A*T*C*A*G*C*T*+C*+C*+A | SEQ ID NO: 6 | 75.90 |
| 100904 | G12D | | | |
| STN- | hKRAS | +T*+A*/iMe- | SEQ ID NO: 10 | 0 |
| 100951 | G12D | dC/*G*C*C*A*T*C*A*G*C*+T*+C*+C | | |
| STN- | hKRAS | +T*+A*/iMe- | SEQ ID NO: 10 | 0 |
| 100952 | G12D | dC/*G*C*C*A*T*C*A*G*C*T*+C*+C | | |
| STN- | hKRAS | +T*+A*+C*G*C*C*A*T*C*A*G*C*T*+C*+C | SEQ ID NO: 10 | 59.00 |
| 100978 | G12D | | | |
| STN- | hKRAS | +T*+A*+C*G*C*C*A*T*C*A*G*C*+T*/iMe- | SEQ ID NO: 10 | 39.90 |
| 100979 | G12D | dC/*+C | | |
| STN- | hKRAS | +T*+A*+C*G*C*C*A*T*C*A*G*C*+T*+C*/ | SEQ ID NO: 10 | 44.70 |
| 100980 | G12D | 3Me-dC/ | | |
| STN- | hKRAS | +A*+C*+G*C*C*A*T*C*A*G*C*T*C*+C*+A | SEQ ID NO: 6 | 52.50 |
| 100983 | G12D | | | |
| STN- | hKRAS | +A*/iMe- | SEQ ID NO: 6 | 64.10 |
| 100984 | G12D | dC/*+G*C*C*A*T*C*A*G*C*T*+C*+C*+A | | |
| STN- | hKRAS | +A*+C*+G*C*C*A*T*C*A*G*C*T*/iMe- | SEQ ID NO: 6 | 61.50 |
| 100985 | G12D | dC/*+C*+A | | |
| STN- | hKRAS | +A*+C*+G* C*C*A* T*C*A* G*C*T* +C*/ | SEQ ID NO: 6 | 61.60 |
| 100986 | G12D | iMe-dC/*+A | | |

“+” indicates LNA, and may be optionally replaced with 2′-MOE or 2′-OMe, or other bridged nucleotide such as cEt;
“*” indicates a phosphorothioate internucleotide linkage;
“iMe” indicates an internucleotide 5-methyl modified nucleotide.
“3Me” indicates a 3′ 5-methyl modified nucleotide.

### Inhibition of Cell Growth:

The ability of ASOs to inhibit cell growth was assessed as follows. Cell lines were plated in clear 384 well plates (S-Bio, #MS-9384UZ) at 800 cells per well in growth medium with 10-20% FBS and 100 IU/mL Pen/Strep overnight. Cells were treated with varying concentrations of ASO. After 7-10 days, cell viability was determined by measuring total ATP content using the Cell Titer Glo reagent (Promega, G7570) according to manufacturer's instructions. The results for growth inhibition are in the table and graph below.

TABLE 9
3D Growth Inhibition of G12V Modified Specific Sequences in CAPAN1 and
LCLC97TM1 Cell Lines

| | | | Sequence | 3D Growth | |
|---|---|---|---|---|---|
| | | | Identifier | Inhibition (%) | |
| | | | for | | LCLC- |
| | | | Nucleobase | Capan-1 | 97TM1 |
| Number | Target | Antisense Oligonucleotide | Sequence | 5 μM | 5 μM |
| STN- | Pan hKRAS | +C*+C*+T*C*A*T*T*G*C*A*C*T*G*+T*+A*+C | SEQ ID NO: | 81.15 | 97.90 |
| 100019 | | | 1 | | |
| STN- | hKRASG12V | +T*+A*+C*G*C*C*A*A*C*A*G*+C*+T*+C | SEQ ID NO: | 69.15 | 91.71 |
| 100080 | | | 2 | | |
| STN- | hKRAS | +T*+A*/iMe-dC/*G*/iMe-dC/*/iMe- | SEQ ID NO: | 4.29 | n/a |
| 100940 | G12V | dC/*A*A*/iMe-dC/*A*G*/iMe-dC/*+T*+C | 2 | | |
| STN- | hKRAS | +T*+A*/iMe-dC/*G*C*C*A*A*C*A*G*/iMe- | SEQ ID NO: | 13.63 | n/a |
| 100941 | G12V | dC/*+T*+C | 2 | | |
| STN- | hKRAS | +T*+A*C*G*C*C*A*A*C*A*G*C*+T*+C | SEQ ID NO: | 14.13 | 5.82 |
| 100942 | G12V | | 2 | | |
| STN- | hKRAS | +T*+A*+C*G*C*C*A*A*C*A*G*C*+T*+C | SEQ ID NO: | n/a | 25.96 |
| 100943 | G12V | | 2 | | |
| STN- | hKRAS | +T*+A*C*G*C*C*A*A*C*A*G*+C*+T*+C | SEQ ID NO: | 49.03 | 71.79 |
| 100944 | G12V | | 2 | | |
| STN- | hKRAS | +C*+T*+A*C*G*C*C*A*A*C*A*+G*+C*+T | SEQ ID NO: | 39.83 | 78.18 |
| 100987 | G12V | | 3 | | |
| STN- | hKRAS | +A*+C*+G*C*C*A*A*C*A*G*C*+T*+C*/3Me- | SEQ ID NO: | 57.41 | 86.80 |
| 100989 | G12V | dC | 5 | | |
| STN- | hKRAS | +A*+C*+G*C*C*A*A*C*A*G*C*+T*/iMe-dC/+C | SEQ ID NO: | 59.39 | 84.55 |
| 100990 | G12V | | 5 | | |
| STN- | hKRAS | +A*/iMe-dC/*G*C*C*A*A*C*A*G*C*+T*+C | SEQ ID NO: | 10.79 | 44.20 |
| 100991 | G12V | | 4 | | |
| STN- | hKRAS | +A*+C*G*C*C*A*A*C*A*G*/iMe-dC/*+T*+C | SEQ ID NO: | 42.12 | 89.10 |
| 100992 | G12V | | 4 | | |
| STN- | hKRAS | +A*+C*G*C*C*A*A*C*A*G*+C*+T*/3Me-dC | SEQ ID NO: | 25.37 | 73.38 |
| 100993 | 312V | | 4 | | |
| STN- | hKRAS | +T*+A*/iMe-dC/*G*C*C*A*A*C*A*G*C+T*+C | SEQ ID NO: | 21.91 | 54.63 |
| 100994 | G12V | | 2 | | |
| STN- | hKRAS | +T*+A*/iMe-dC/*G*C*C*A*A*C*A*G+C*+T*+C | SEQ ID NO: | 35.55 | 79.25 |
| 100995 | G12V | | 2 | | |
| STN- | hKRAS | +T*+A*+C*G*C*C*A*A*C*A*G+C*+T*/3Me-dC | SEQ ID NO: | 26.51 | 87.02 |
| 100996 | G12V | | 2 | | |
| STN- | hKRAS | +A*/iMe- | SEQ ID NO: | 6.04 | 25.44 |
| 101007 | G12V | dC/*+G*C*C*A*T*C*A*G*C*T*+C*/iMe-dC/*+A | 6 | | |

“+” indicates LNA, and may be optionally replaced with 2′-MOE or 2′-OMe, or other bridged nucleotide such as cEt;
“*” indicates a phosphorothioate internucleotide linkage;
“iMe” indicates an internucleotide 5-methyl modified nucleotide.
“3Me” indicates a 3′ 5-methyl modified nucleotide.

TABLE 10
3D Growth Inhibition of G12C Modified Specific
Sequences in MIA PaCa-2 Cell Line

| | | | Sequence | 3D Growth |
|---|---|---|---|---|
| | | | Identifier for | Inhibition (%) |
| | | | Nucleobase | MIA PaCa-2 |
| Number | Target | Antisense Oligonucleotide | Sequence | 5 μM |
| STN- | Pan | +C*+C*+T*C*A*T*T*G*C*A*C*T*G*+T*+A*+C | SEQ ID NO: 1 | 85.30 |
| 100019 | hKRAS | | | |
| STN- | hKRAS | +T*+A*+C*G*C*C*A*C*A*A*G*+C*+T*+C | SEQ ID NO: 7 | 67.10 |
| 100840 | G12C | | | |
| STN- | hKRAS | +A*+C*+G*C*C*A*C*A*A*G*C*+T*+C | SEQ ID NO: 8 | 66.15 |
| 100841 | G12C | | | |
| STN- | hKRAS | +A*+C*G*C*C*A*C*A*A*G*+C*+T*+C | SEQ ID NO: 8 | 51.00 |
| 100842 | G12C | | | |
| STN- | hKRAS | +A*+C*+G*C*C*A*C*A*A*G*/i5HydMe- | SEQ ID NO: 9 | 11.05 |
| 100843 | G12C | dC/*+T*+C*+C | | |
| STN- | hKRAS | +A*+C*+G*C*/12MOErC/*A*C*A*A*G*C*+T*+C*+C | SEQ ID NO: 9 | 13.65 |
| 100844 | G12C | | | |
| STN- | hKRAS | +A*+C*+G*C*/12MOErC/*A*C*A*A*G*/i5HydMe- | SEQ ID NO: 9 | 3.85 |
| 100845 | G12C | dC/*+T*+C*+C | | |
| STN- | hKRAS | +A*+C*+G*C*C*A*C*A*A*/12MOErG/*C*+T*+C*+C | SEQ ID NO: 9 | 16.10 |
| 100846 | G12C | | | |
| STN- | hKRAS | +T*+A*+/iMe-dC/*G*C*C*A*C*A*A*G*+C*+T*+C | SEQ ID NO: 7 | 74.58 |
| 100949 | G12C | | | |
| STN- | hKRAS | +T*+A*+/iMe-dC/*G*C*C*A*C*A*A*G*C*+T*+C | SEQ ID NO: 7 | 72.84 |
| 100950 | G12C | | | |

“+” indicates LNA, and may be optionally replaced with 2′-MOE or 2′-OMe, or other bridged nucleotide such as cEt;
“*” indicates a phosphorothioate internucleotide linkage;
“i2MOEr” indicates an internucleotide 2-MethoxyEthoxy modified nucleotide;
“i5HydMe” indicates an internucleotide 5-hydroxymethyl modified nucleotide.
“iMe” indicates an internucleotide 5-methyl modified nucleotide.

TABLE 11
3D Growth Inhibition of G12D Modified Specific
Sequences in PANC-1 Cell Line

| | | | Sequence | 3D Growth |
|---|---|---|---|---|
| | | | Identifier for | Inhibition (%) |
| | | | Nucleobase | PANC-1 |
| Number | Target | Antisense Oligonucleotide | Sequence | 5 μM |
| STN- | Pan | +C*+C*+T*C*A*T*T*G*C*A*C* | SEQ ID NO: 1 | 95.00 |
| 100019 | hKRAS | T*G*+T*+A*+C | | |
| STN- | hKRAS | +T*+A*+C*G*C*C*A*T*C*A*G* | SEQ ID NO: 10 | 55.40 |
| 100903 | G12D | C*+T*+C*+C | | |
| STN- | hKRAS | +A*+C*+G*C*C*A*T*C*A*G*C* | SEQ ID NO: 6 | 75.90 |
| 100904 | G12D | T*+C*+C*+A | | |
| STN- | hKRAS | +T*+A*/iMe-dC/*G*C* | SEQ ID NO: 10 | 0 |
| 100951 | G12D | C*A*T*C*A*G*C*+T*+C*+C | | |
| STN- | hKRAS | +T*+A*/iMe-dC/*G*C*C* | SEQ ID NO: 10 | 0 |
| 100952 | G12D | A*T*C*A*G*C*T*+C*+C | | |
| STN- | hKRAS | +T*+A*+C*G*C*C*A*T*C*A*G* | SEQ ID NO: 10 | 59.00 |
| 100978 | G12D | C*T*+C*+C | | |
| STN- | hKRAS | +T*+A*+C*G*C*C*A*T*C*A*G* | SEQ ID NO: 10 | 39.90 |
| 100979 | G12D | C*+T*/iMe-dC/*+C | | |
| STN- | hKRAS | +T*+A*+C*G*C*C*A*T*C*A*G* | SEQ ID NO: 10 | 44.70 |
| 100980 | G12D | C*+T*+C*/3Me-dC/ | | |
| STN- | hKRAS | +A*+C*+G*C*C*A*T*C*A*G*C* | SEQ ID NO: 6 | 52.50 |
| 100983 | G12D | T*C*+C*+A | | |
| STN- | hKRAS | +A*/iMe-dC/*+G*C*C*A* | SEQ ID NO: 6 | 64.10 |
| 100984 | G12D | T*C*A*G*C*T*+C*+C*+A | | |
| STN- | hKRAS | +A*+C*+G*C*C*A*T*C*A*G*C* | SEQ ID NO: 6 | 61.50 |
| 100985 | G12D | T*/iMe-dC/*+C*+A | | |
| STN- | hKRAS | +A*+C*+G* C*C*A* T*C*A* | SEQ ID NO: 6 | 61.60 |
| 100986 | G12D | G*C*T* +C*/iMe-dC/*+A | | |

“+” indicates LNA, and may be optionally replaced with 2′-MOE or 2′-OMe, or other bridged nucleotide such as cEt;
“*” indicates a phosphorothioate internucleotide linkage;
“iMe” indicates an internucleotide 5-methyl modified nucleotide.
“3Me” indicates a 3′ 5-methyl modified nucleotide.

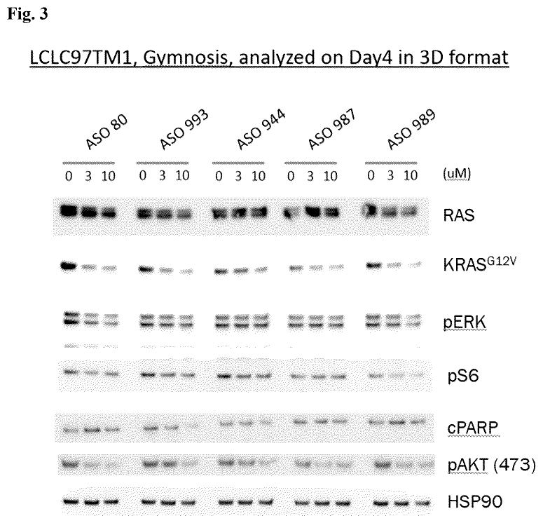

FIG. 3 shows a Western Blot analysis after G12V KRAS knockdown with ASOs in LCLC-97TM1 Cell Line (Day 4). FIG. 3 demonstrates knockdown of KRAS protein and pathway modulation.

TABLE 4
Wildtype or mutated KRAS mRNA sequences

| SEQ ID | KRAS | |
|---|---|---|
| NO | mRNA | Sequence |
| SEQ ID | Wildtype | CTAGGCGGCGGCCGCGGCGGCGGAGGCAGCAGCGGCGGCGGCAGTGGCGGC |
| NO: 11 | (GenBank | GGCGAAGGTGGCGGCGGCTCGGCCAGTACTCCCGGCCCCCGCCATTTCGGAC |
| | Reference | TGGGAGCGAGCGCGGCGCAGGCACTGAAGGCGGCGGCGGGGCCAGAGGC |
| | number: | TCAGCGGCTCCCAGGTGCGGGAGAGAGGCCTGCTGAAAATGACTGAATATAA |
| | NM_033360.4) | ACTTGTGGTAGTTGGAGCTGGTGGCGTAGGCAAGAGTGCCTTGACGATACAG |
| | | CTAATTCAGAATCATTTTGTGGACGAATATGATCCAACAATAGAGGATTCCTA |
| | | CAGGAAGCAAGTAGTAATTGATGGAGAAACCTGTCTCTTGGATATTCTCGACA |
| | | CAGCAGGTCAAGAGGAGTACAGTGCAATGAGGGACCAGTACATGAGGACTG |
| | | GGGAGGGCTTTCTTTGTGTATTTGCCATAAATAATACTAAATCATTTGAAGAT |
| | | ATTCACCATTATAGAGAACAAATTAAAAGAGTTAAGGACTCTGAAGATGTAC |
| | | CTATGGTCCTAGTAGGAAATAAATGTGATTTGCCTTCTAGAACAGTAGACACA |
| | | AAACAGGCTCAGGACTTAGCAAGAAGTTATGGAATTCCTTTTATTGAAACATC |
| | | AGCAAAGACAAGACAGAGAGTGGAGGATGCTTTTTATACATTGGTGAGAGAG |
| | | ATCCGACAATACAGATTGAAAAAAATCAGCAAAGAAGAAAAGACTCCTGGCT |
| | | GTGTGAAAATTAAAAAATGCATTATAATGTAATCTGGGTGTTGATGATGCCTT |
| | | CTATACATTAGTTCGAGAAATTCGAAAACATAAAGAAAAGATGAGCAAAGAT |
| | | GGTAAAAAGAAGAAAAAGAAGTCAAAGACAAAGTGTGTAATTATGTAAATA |
| | | CAATTTGTACTTTTTTCTTAAGGCATACTAGTACAAGTGGTAATTTTTGTACAT |
| | | TACACTAAATTATTAGCATTTGTTTTAGCATTACCTAATTTTTTTCCTGCTCCAT |
| | | GCAGACTGTTAGCTTTTACCTTAAATGCTTATTTTAAAATGACAGTGGAAGTT |
| | | TTTTTTTCCTCTAAGTGCCAGTATTCCCAGAGTTTTGGTTTTTGAACTAGCAAT |
| | | GCCTGTGAAAAAGAAACTGAATACCTAAGATTTCTGTCTTGGGGCTTTTGGTG |
| | | CATGCAGTTGATTACTTCTTATTTTTCTTACCAATTGTGAATGTTGGTGTGAAA |
| | | CAAATTAATGAAGCTTTTGAATCATCCCTATTCTGTGTTTTATCTAGTCACATA |
| | | AATGGATTAATTACTAATTTCAGTTGAGACCTTCTAATTGGTTTTTACTGAAAC |
| | | ATTGAGGGAACACAAATTTATGGGCTTCCTGATGATGATTCTTCTAGGCATCA |
| | | TGTCCTATAGTTTGTCATCCCTGATGAATGTAAAGTTACACTGTTCACAAAGG |
| | | TTTTGTCTCCTTTCCACTGCTATTAGTCATGGTCACTCTCCCCAAAATATTATA |
| | | TTTTTTCTATAAAAAGAAAAAAATGGAAAAAAATTACAAGGCAATGGAAACT |
| | | ATTATAAGGCCATTTCCTTTTCACATTAGATAAATTACTATAAAGACTCCTAAT |
| | | AGCTTTTCCTGTTAAGGCAGACCCAGTATGAAATGGGGATTATTATAGCAACC |
| | | ATTTTGGGGCTATATTTACATGCTACTAAATTTTTATAATAATTGAAAAGATTT |
| | | TAACAAGTATAAAAAATTCTCATAGGAATTAAATGTAGTCTCCCTGTGTCAGA |
| | | CTGCTCTTTCATAGTATAACTTTAAATCTTTTCTTCAACTTGAGTCTTTGAAGA |
| | | TAGTTTTAATTCTGCTTGTGACATTAAAAGATTATTTGGGCCAGTTATAGCTTA |
| | | TTAGGTGTTGAAGAGACCAAGGTTGCAAGGCCAGGCCCTGTGTGAACCTTTG |
| | | AGCTTTCATAGAGAGTTTCACAGCATGGACTGTGTCCCCACGGTCATCCAGTG |
| | | TTGTCATGCATTGGTTAGTCAAAATGGGGAGGGACTAGGGCAGTTTGGATAG |
| | | CTCAACAAGATACAATCTCACTCTGTGGTGGTCCTGCTGACAAATCAAGAGCA |
| | | TTGCTTTTGTTTCTTAAGAAAACAAACTCTTTTTTAAAAATTACTTTTAAATAT |
| | | TAACTCAAAAGTTGAGATTTTGGGGTGGTGGTGTGCCAAGACATTAATTTTTT |
| | | TTTTAAACAATGAAGTGAAAAAGTTTTACAATCTCTAGGTTTGGCTAGTTCTC |
| | | TTAACACTGGTTAAATTAACATTGCATAAACACTTTTCAAGTCTGATCCATATT |
| | | TAATAATGCTTTAAAATAAAAATAAAAACAATCCTTTTGATAAATTTAAAATG |
| | | TTACTTATTTTAAAATAAATGAAGTGAGATGGCATGGTGAGGTGAAAGTATCA |
| | | CTGGACTAGGAAGAAGGTGACTTAGGTTCTAGATAGGTGTCTTTTAGGACTCT |
| | | GATTTTGAGGACATCACTTACTATCCATTTCTTCATGTTAAAAGAAGTCATCTC |
| | | AAACTCTTAGTTTTTTTTTTTTACAACTATGTAATTTATATTCCATTTACATAAG |
| | | GATACACTTATTTGTCAAGCTCAGCACAATCTGTAAATTTTTAACCTATGTTAC |
| | | ACCATCTTCAGTGCCAGTCTTGGGCAAAATTGTGCAAGAGGTGAAGTTTATAT |
| | | TTGAATATCCATTCTCGTTTTAGGACTCTTCTTCCATATTAGTGTCATCTTGCCT |
| | | CCCTACCTTCCACATGCCCCATGACTTGATGCAGTTTTAATACTTGTAATTCCC |
| | | CTAACCATAAGATTTACTGCTGCTGTGGATATCTCCATGAAGTTTTCCCACTG |
| | | AGTCACATCAGAAATGCCCTACATCTTATTTCCTCAGGGCTCAAGAGAATCTG |
| | | ACAGATACCATAAAGGGATTTGACCTAATCACTAATTTTCAGGTGGTGGCTGA |
| | | TGCTTTGAACATCTCTTTGCTGCCCAATCCATTAGCGACAGTAGGATTTTTCAA |
| | | ACCTGGTATGAATAGACAGAACCCTATCCAGTGGAAGGAGAATTTAATAAAG |
| | | ATAGTGCTGAAAGAATTCCTTAGGTAATCTATAACTAGGACTACTCCTGGTAA |
| | | CAGTAATACATTCCATTGTTTTAGTAACCAGAAATCTTCATGCAATGAAAAAT |
| | | ACTTTAATTCATGAAGCTTACTTTTTTTTTTTGGTGTCAGAGTCTCGCTCTTGTC |
| | | ACCCAGGCTGGAATGCAGTGGCGCCATCTCAGCTCACTGCAACCTCCATCTCC |
| | | CAGGTTCAAGCGATTCTCGTGCCTCGGCCTCCTGAGTAGCTGGGATTACAGGC |
| | | GTGTGCCACTACACTCAACTAATTTTTGTATTTTTAGGAGAGACGGGGTTTCA |
| | | CCCTGTTGGCCAGGCTGGTCTCGAACTCCTGACCTCAAGTGATTCACCCACCT |
| | | TGGCCTCATAAACCTGTTTTGCAGAACTCATTTATTCAGCAAATATTTATTGAG |
| | | TGCCTACCAGATGCCAGTCACCACACAAGGCACTGGGTATATGGTATCCCCAA |
| | | ACAAGAGACATAATCCCGGTCCTTAGGTAGTGCTAGTGTGGTCTGTAATATCT |
| | | TACTAAGGCCTTTGGTATACGACCCAGAGATAACACGATGCGTATTTTAGTTT |
| | | TGCAAAGAAGGGGTTTGGTCTCTGTGCCAGCTCTATAATTGTTTTGCTACGAT |
| | | TCCACTGAAACTCTTCGATCAAGCTACTTTATGTAAATCACTTCATTGTTTTAA |
| | | AGGAATAAACTTGATTATATTGTTTTTTTATTTGGCATAACTGTGATTCTTTTA |
| | | GGACAATTACTGTACACATTAAGGTGTATGTCAGATATTCATATTGACCCAAA |
| | | TGTGTAATATTCCAGTTTTCTCTGCATAAGTAATTAAAATATACTTAAAAATTA |
| | | ATAGTTTTATCTGGGTACAAATAAACAGGTGCCTGAACTAGTTCACAGACAAG |
| | | GAAACTTCTATGTAAAAATCACTATGATTTCTGAATTGCTATGTGAAACTACA |
| | | GATCTTTGGAACACTGTTTAGGTAGGGTGTTAAGACTTACACAGTACCTCGTT |
| | | TCTACACAGAGAAAGAAATGGCCATACTTCAGGAACTGCAGTGCTTATGAGG |
| | | GGATATTTAGGCCTCTTGAATTTTTGATGTAGATGGGCATTTTTTTAAGGTAGT |
| | | GGTTAATTACCTTTATGTGAACTTTGAATGGTTTAACAAAAGATTTGTTTTTGT |
| | | AGAGATTTTAAAGGGGGAGAATTCTAGAAATAAATGTTACCTAATTATTACA |
| | | GCCTTAAAGACAAAAATCCTTGTTGAAGTTTTTTTAAAAAAAGCTAAATTACA |
| | | TAGACTTAGGCATTAACATGTTTGTGGAAGAATATAGCAGACGTATATTGTAT |
| | | CATTTGAGTGAATGTTCCCAAGTAGGCATTCTAGGCTCTATTTAACTGAGTCA |
| | | CACTGCATAGGAATTTAGAACCTAACTTTTATAGGTTATCAAAACTGTTGTCA |
| | | CCATTGCACAATTTTGTCCTAATATATACATAGAAACTTTGTGGGGCATGTTA |
| | | AGTTACAGTTTGCACAAGTTCATCTCATTTGTATTCCATTGATTTTTTTTTTCTT |
| | | CTAAACATTTTTTCTTCAAACAGTATATAACTTTTTTTAGGGGATTTTTTTTTA |
| | | GACAGCAAAAACTATCTGAAGATTTCCATTTGTCAAAAAGTAATGATTTCTTG |
| | | ATAATTGTGTAGTAATGTTTTTTAGAACCCAGCAGTTACCTTAAAGCTGAATT |
| | | TATATTTAGTAACTTCTGTGTTAATACTGGATAGCATGAATTCTGCATTGAGA |
| | | AACTGAATAGCTGTCATAAAATGAAACTTTCTTTCTAAAGAAAGATACTCACA |
| | | TG |
| SEQ ID | G12A | CTAGGCGGCGGCCGCGGCGGCGGAGGCAGCAGCGGCGGCGGCAGTGGCGGC |
| NO: 12 | | GGCGAAGGTGGCGGCGGCTCGGCCAGTACTCCCGGCCCCCGCCATTTCGGAC |
| | | TGGGAGCGAGCGCGGCGCAGGCACTGAAGGCGGCGGCGGGGCCAGAGGCTC |
| | | AGCGGCTCCCAGGTGCGGGAGAGAGGCCTGCTGAAAATGACTGAATATAAAC |
| | | TTGTGGTAGTTGGAGCTGCTGGCGTAGGCAAGAGTGCCTTGACGATACAGCTA |
| | | ATTCAGAATCATTTTGTGGACGAATATGATCCAACAATAGAGGATTCCTACAG |
| | | GAAGCAAGTAGTAATTGATGGAGAAACCTGTCTCTTGGATATTCTCGACACAG |
| | | CAGGTCAAGAGGAGTACAGTGCAATGAGGGACCAGTACATGAGGACTGGGG |
| | | AGGGCTTTCTTTGTGTATTTGCCATAAATAATACTAAATCATTTGAAGATATTC |
| | | ACCATTATAGAGAACAAATTAAAAGAGTTAAGGACTCTGAAGATGTACCTAT |
| | | GGTCCTAGTAGGAAATAAATGTGATTTGCCTTCTAGAACAGTAGACACAAAA |
| | | CAGGCTCAGGACTTAGCAAGAAGTTATGGAATTCCTTTTATTGAAACATCAGC |
| | | AAAGACAAGACAGAGAGTGGAGGATGCTTTTTATACATTGGTGAGAGAGATC |
| | | CGACAATACAGATTGAAAAAAATCAGCAAAGAAGAAAAGACTCCTGGCTGTG |
| | | TGAAAATTAAAAAATGCATTATAATGTAATCTGGGTGTTGATGATGCCTTCTA |
| | | TACATTAGTTCGAGAAATTCGAAAACATAAAGAAAAGATGAGCAAAGATGGT |
| | | AAAAAGAAGAAAAAGAAGTCAAAGACAAAGTGTGTAATTATGTAAATACAA |
| | | TTTGTACTTTTTTCTTAAGGCATACTAGTACAAGTGGTAATTTTTGTACATTAC |
| | | ACTAAATTATTAGCATTTGTTTTAGCATTACCTAATTTTTTTCCTGCTCCATGC |
| | | AGACTGTTAGCTTTTACCTTAAATGCTTATTTTAAAATGACAGTGGAAGTTTTT |
| | | TTTTCCTCTAAGTGCCAGTATTCCCAGAGTTTTGGTTTTTGAACTAGCAATGCC |
| | | TGTGAAAAAGAAACTGAATACCTAAGATTTCTGTCTTGGGGCTTTTGGTGCAT |
| | | GCAGTTGATTACTTCTTATTTTTCTTACCAATTGTGAATGTTGGTGTGAAACAA |
| | | ATTAATGAAGCTTTTGAATCATCCCTATTCTGTGTTTTATCTAGTCACATAAAT |
| | | GGATTAATTACTAATTTCAGTTGAGACCTTCTAATTGGTTTTTACTGAAACATT |
| | | GAGGGAACACAAATTTATGGGCTTCCTGATGATGATTCTTCTAGGCATCATGT |
| | | CCTATAGTTTGTCATCCCTGATGAATGTAAAGTTACACTGTTCACAAAGGTTTT |
| | | GTCTCCTTTCCACTGCTATTAGTCATGGTCACTCTCCCCAAAATATTATATTTT |
| | | TTCTATAAAAAGAAAAAAATGGAAAAAAATTACAAGGCAATGGAAACTATTA |
| | | TAAGGCCATTTCCTTTTCACATTAGATAAATTACTATAAAGACTCCTAATAGC |
| | | TTTTCCTGTTAAGGCAGACCCAGTATGAAATGGGGATTATTATAGCAACCATT |
| | | TTGGGGCTATATTTACATGCTACTAAATTTTTATAATAATTGAAAAGATTTTAA |
| | | CAAGTATAAAAAATTCTCATAGGAATTAAATGTAGTCTCCCTGTGTCAGACTG |
| | | CTCTTTCATAGTATAACTTTAAATCTTTTCTTCAACTTGAGTCTTTGAAGATAG |
| | | TTTTAATTCTGCTTGTGACATTAAAAGATTATTTGGGCCAGTTATAGCTTATTA |
| | | GGTGTTGAAGAGACCAAGGTTGCAAGGCCAGGCCCTGTGTGAACCTTTGAGC |
| | | TTTCATAGAGAGTTTCACAGCATGGACTGTGTCCCCACGGTCATCCAGTGTTG |
| | | TCATGCATTGGTTAGTCAAAATGGGGAGGGACTAGGGCAGTTTGGATAGCTC |
| | | AACAAGATACAATCTCACTCTGTGGTGGTCCTGCTGACAAATCAAGAGCATTG |
| | | CTTTTGTTTCTTAAGAAAACAAACTCTTTTTTAAAAATTACTTTTAAATATTAA |
| | | CTCAAAAGTTGAGATTTTGGGGTGGTGGTGTGCCAAGACATTAATTTTTTTTTT |
| | | AAACAATGAAGTGAAAAAGTTTTACAATCTCTAGGTTTGGCTAGTTCTCTTAA |
| | | CACTGGTTAAATTAACATTGCATAAACACTTTTCAAGTCTGATCCATATTTAAT |
| | | AATGCTTTAAAATAAAAATAAAAACAATCCTTTTGATAAATTTAAAATGTTAC |
| | | TTATTTTAAAATAAATGAAGTGAGATGGCATGGTGAGGTGAAAGTATCACTG |
| | | GACTAGGAAGAAGGTGACTTAGGTTCTAGATAGGTGTCTTTTAGGACTCTGAT |
| | | TTTGAGGACATCACTTACTATCCATTTCTTCATGTTAAAAGAAGTCATCTCAA |
| | | ACTCTTAGTTTTTTTTTTTTACAACTATGTAATTTATATTCCATTTACATAAGGA |
| | | TACACTTATTTGTCAAGCTCAGCACAATCTGTAAATTTTTAACCTATGTTACAC |
| | | CATCTTCAGTGCCAGTCTTGGGCAAAATTGTGCAAGAGGTGAAGTTTATATTT |
| | | GAATATCCATTCTCGTTTTAGGACTCTTCTTCCATATTAGTGTCATCTTGCCTC |
| | | CCTACCTTCCACATGCCCCATGACTTGATGCAGTTTTAATACTTGTAATTCCCC |
| | | TAACCATAAGATTTACTGCTGCTGTGGATATCTCCATGAAGTTTTCCCACTGA |
| | | GTCACATCAGAAATGCCCTACATCTTATTTCCTCAGGGCTCAAGAGAATCTGA |
| | | CAGATACCATAAAGGGATTTGACCTAATCACTAATTTTCAGGTGGTGGCTGAT |
| | | GCTTTGAACATCTCTTTGCTGCCCAATCCATTAGCGACAGTAGGATTTTTCAA |
| | | ACCTGGTATGAATAGACAGAACCCTATCCAGTGGAAGGAGAATTTAATAAAG |
| | | ATAGTGCTGAAAGAATTCCTTAGGTAATCTATAACTAGGACTACTCCTGGTAA |
| | | CAGTAATACATTCCATTGTTTTAGTAACCAGAAATCTTCATGCAATGAAAAAT |
| | | ACTTTAATTCATGAAGCTTACTTTTTTTTTTTGGTGTCAGAGTCTCGCTCTTGTC |
| | | ACCCAGGCTGGAATGCAGTGGCGCCATCTCAGCTCACTGCAACCTCCATCTCC |
| | | CAGGTTCAAGCGATTCTCGTGCCTCGGCCTCCTGAGTAGCTGGGATTACAGGC |
| | | GTGTGCCACTACACTCAACTAATTTTTGTATTTTTAGGAGAGACGGGGTTTCA |
| | | CCCTGTTGGCCAGGCTGGTCTCGAACTCCTGACCTCAAGTGATTCACCCACCT |
| | | TGGCCTCATAAACCTGTTTTGCAGAACTCATTTATTCAGCAAATATTTATTGAG |
| | | TGCCTACCAGATGCCAGTCACCACACAAGGCACTGGGTATATGGTATCCCCAA |
| | | ACAAGAGACATAATCCCGGTCCTTAGGTAGTGCTAGTGTGGTCTGTAATATCT |
| | | TACTAAGGCCTTTGGTATACGACCCAGAGATAACACGATGCGTATTTTAGTTT |
| | | TGCAAAGAAGGGGTTTGGTCTCTGTGCCAGCTCTATAATTGTTTTGCTACGAT |
| | | TCCACTGAAACTCTTCGATCAAGCTACTTTATGTAAATCACTTCATTGTTTTAA |
| | | AGGAATAAACTTGATTATATTGTTTTTTTATTTGGCATAACTGTGATTCTTTTA |
| | | GGACAATTACTGTACACATTAAGGTGTATGTCAGATATTCATATTGACCCAAA |
| | | TGTGTAATATTCCAGTTTTCTCTGCATAAGTAATTAAAATATACTTAAAAATTA |
| | | ATAGTTTTATCTGGGTACAAATAAACAGGTGCCTGAACTAGTTCACAGACAAG |
| | | GAAACTTCTATGTAAAAATCACTATGATTTCTGAATTGCTATGTGAAACTACA |
| | | GATCTTTGGAACACTGTTTAGGTAGGGTGTTAAGACTTACACAGTACCTCGTT |
| | | TCTACACAGAGAAAGAAATGGCCATACTTCAGGAACTGCAGTGCTTATGAGG |
| | | GGATATTTAGGCCTCTTGAATTTTTGATGTAGATGGGCATTTTTTTAAGGTAGT |
| | | GGTTAATTACCTTTATGTGAACTTTGAATGGTTTAACAAAAGATTTGTTTTTGT |
| | | AGAGATTTTAAAGGGGGAGAATTCTAGAAATAAATGTTACCTAATTATTACA |
| | | GCCTTAAAGACAAAAATCCTTGTTGAAGTTTTTTTAAAAAAAGCTAAATTACA |
| | | TAGACTTAGGCATTAACATGTTTGTGGAAGAATATAGCAGACGTATATTGTAT |
| | | CATTTGAGTGAATGTTCCCAAGTAGGCATTCTAGGCTCTATTTAACTGAGTCA |
| | | CACTGCATAGGAATTTAGAACCTAACTTTTATAGGTTATCAAAACTGTTGTCA |
| | | CCATTGCACAATTTTGTCCTAATATATACATAGAAACTTTGTGGGGCATGTTA |
| | | AGTTACAGTTTGCACAAGTTCATCTCATTTGTATTCCATTGATTTTTTTTTTCTT |
| | | CTAAACATTTTTTCTTCAAACAGTATATAACTTTTTTTAGGGGATTTTTTTTTA |
| | | GACAGCAAAAACTATCTGAAGATTTCCATTTGTCAAAAAGTAATGATTTCTTG |
| | | ATAATTGTGTAGTAATGTTTTTTAGAACCCAGCAGTTACCTTAAAGCTGAATT |
| | | TATATTTAGTAACTTCTGTGTTAATACTGGATAGCATGAATTCTGCATTGAGA |
| | | AACTGAATAGCTGTCATAAAATGAAACTTTCTTTCTAAAGAAAGATACTCACA |
| | | TG |
| SEQ ID | G12C | CTAGGCGGCGGCCGCGGCGGCGGAGGCAGCAGCGGCGGCGGCAGTGGCGGC |
| NO: 13 | | GGCGAAGGTGGCGGCGGCTCGGCCAGTACTCCCGGCCCCCGCCATTTCGGAC |
| | | TGGGAGCGAGCGCGGCGCAGGCACTGAAGGCGGCGGCGGGGCCAGAGGCTC |
| | | AGCGGCTCCCAGGTGCGGGAGAGAGGCCTGCTGAAAATGACTGAATATAAAC |
| | | TTGTGGTAGTTGGAGCTTGTGGCGTAGGCAAGAGTGCCTTGACGATACAGCTA |
| | | ATTCAGAATCATTTTGTGGACGAATATGATCCAACAATAGAGGATTCCTACAG |
| | | GAAGCAAGTAGTAATTGATGGAGAAACCTGTCTCTTGGATATTCTCGACACAG |
| | | CAGGTCAAGAGGAGTACAGTGCAATGAGGGACCAGTACATGAGGACTGGGG |
| | | AGGGCTTTCTTTGTGTATTTGCCATAAATAATACTAAATCATTTGAAGATATTC |
| | | ACCATTATAGAGAACAAATTAAAAGAGTTAAGGACTCTGAAGATGTACCTAT |
| | | GGTCCTAGTAGGAAATAAATGTGATTTGCCTTCTAGAACAGTAGACACAAAA |
| | | CAGGCTCAGGACTTAGCAAGAAGTTATGGAATTCCTTTTATTGAAACATCAGC |
| | | AAAGACAAGACAGAGAGTGGAGGATGCTTTTTATACATTGGTGAGAGAGATC |
| | | CGACAATACAGATTGAAAAAAATCAGCAAAGAAGAAAAGACTCCTGGCTGTG |
| | | TGAAAATTAAAAAATGCATTATAATGTAATCTGGGTGTTGATGATGCCTTCTA |
| | | TACATTAGTTCGAGAAATTCGAAAACATAAAGAAAAGATGAGCAAAGATGGT |
| | | AAAAAGAAGAAAAAGAAGTCAAAGACAAAGTGTGTAATTATGTAAATACAA |
| | | TTTGTACTTTTTTCTTAAGGCATACTAGTACAAGTGGTAATTTTTGTACATTAC |
| | | ACTAAATTATTAGCATTTGTTTTAGCATTACCTAATTTTTTTCCTGCTCCATGC |
| | | AGACTGTTAGCTTTTACCTTAAATGCTTATTTTAAAATGACAGTGGAAGTTTTT |
| | | TTTTCCTCTAAGTGCCAGTATTCCCAGAGTTTTGGTTTTTGAACTAGCAATGCC |
| | | TGTGAAAAAGAAACTGAATACCTAAGATTTCTGTCTTGGGGCTTTTGGTGCAT |
| | | GCAGTTGATTACTTCTTATTTTTCTTACCAATTGTGAATGTTGGTGTGAAACAA |
| | | ATTAATGAAGCTTTTGAATCATCCCTATTCTGTGTTTTATCTAGTCACATAAAT |
| | | GGATTAATTACTAATTTCAGTTGAGACCTTCTAATTGGTTTTTACTGAAACATT |
| | | GAGGGAACACAAATTTATGGGCTTCCTGATGATGATTCTTCTAGGCATCATGT |
| | | CCTATAGTTTGTCATCCCTGATGAATGTAAAGTTACACTGTTCACAAAGGTTTT |
| | | GTCTCCTTTCCACTGCTATTAGTCATGGTCACTCTCCCCAAAATATTATATTTT |
| | | TTCTATAAAAAGAAAAAAATGGAAAAAAATTACAAGGCAATGGAAACTATTA |
| | | TAAGGCCATTTCCTTTTCACATTAGATAAATTACTATAAAGACTCCTAATAGC |
| | | TTTTCCTGTTAAGGCAGACCCAGTATGAAATGGGGATTATTATAGCAACCATT |
| | | TTGGGGCTATATTTACATGCTACTAAATTTTTATAATAATTGAAAAGATTTTAA |
| | | CAAGTATAAAAAATTCTCATAGGAATTAAATGTAGTCTCCCTGTGTCAGACTG |
| | | CTCTTTCATAGTATAACTTTAAATCTTTTCTTCAACTTGAGTCTTTGAAGATAG |
| | | TTTTAATTCTGCTTGTGACATTAAAAGATTATTTGGGCCAGTTATAGCTTATTA |
| | | GGTGTTGAAGAGACCAAGGTTGCAAGGCCAGGCCCTGTGTGAACCTTTGAGC |
| | | TTTCATAGAGAGTTTCACAGCATGGACTGTGTCCCCACGGTCATCCAGTGTTG |
| | | TCATGCATTGGTTAGTCAAAATGGGGAGGGACTAGGGCAGTTTGGATAGCTC |
| | | AACAAGATACAATCTCACTCTGTGGTGGTCCTGCTGACAAATCAAGAGCATTG |
| | | CTTTTGTTTCTTAAGAAAACAAACTCTTTTTTAAAAATTACTTTTAAATATTAA |
| | | CTCAAAAGTTGAGATTTTGGGGTGGTGGTGTGCCAAGACATTAATTTTTTTTTT |
| | | AAACAATGAAGTGAAAAAGTTTTACAATCTCTAGGTTTGGCTAGTTCTCTTAA |
| | | CACTGGTTAAATTAACATTGCATAAACACTTTTCAAGTCTGATCCATATTTAAT |
| | | AATGCTTTAAAATAAAAATAAAAACAATCCTTTTGATAAATTTAAAATGTTAC |
| | | TTATTTTAAAATAAATGAAGTGAGATGGCATGGTGAGGTGAAAGTATCACTG |
| | | GACTAGGAAGAAGGTGACTTAGGTTCTAGATAGGTGTCTTTTAGGACTCTGAT |
| | | TTTGAGGACATCACTTACTATCCATTTCTTCATGTTAAAAGAAGTCATCTCAA |
| | | ACTCTTAGTTTTTTTTTTTTACAACTATGTAATTTATATTCCATTTACATAAGGA |
| | | TACACTTATTTGTCAAGCTCAGCACAATCTGTAAATTTTTAACCTATGTTACAC |
| | | CATCTTCAGTGCCAGTCTTGGGCAAAATTGTGCAAGAGGTGAAGTTTATATTT |
| | | GAATATCCATTCTCGTTTTAGGACTCTTCTTCCATATTAGTGTCATCTTGCCTC |
| | | CCTACCTTCCACATGCCCCATGACTTGATGCAGTTTTAATACTTGTAATTCCCC |
| | | TAACCATAAGATTTACTGCTGCTGTGGATATCTCCATGAAGTTTTCCCACTGA |
| | | GTCACATCAGAAATGCCCTACATCTTATTTCCTCAGGGCTCAAGAGAATCTGA |
| | | CAGATACCATAAAGGGATTTGACCTAATCACTAATTTTCAGGTGGTGGCTGAT |
| | | GCTTTGAACATCTCTTTGCTGCCCAATCCATTAGCGACAGTAGGATTTTTCAA |
| | | ACCTGGTATGAATAGACAGAACCCTATCCAGTGGAAGGAGAATTTAATAAAG |
| | | ATAGTGCTGAAAGAATTCCTTAGGTAATCTATAACTAGGACTACTCCTGGTAA |
| | | CAGTAATACATTCCATTGTTTTAGTAACCAGAAATCTTCATGCAATGAAAAAT |
| | | ACTTTAATTCATGAAGCTTACTTTTTTTTTTTGGTGTCAGAGTCTCGCTCTTGTC |
| | | ACCCAGGCTGGAATGCAGTGGCGCCATCTCAGCTCACTGCAACCTCCATCTCC |
| | | CAGGTTCAAGCGATTCTCGTGCCTCGGCCTCCTGAGTAGCTGGGATTACAGGC |
| | | GTGTGCCACTACACTCAACTAATTTTTGTATTTTTAGGAGAGACGGGGTTTCA |
| | | CCCTGTTGGCCAGGCTGGTCTCGAACTCCTGACCTCAAGTGATTCACCCACCT |
| | | TGGCCTCATAAACCTGTTTTGCAGAACTCATTTATTCAGCAAATATTTATTGAG |
| | | TGCCTACCAGATGCCAGTCACCACACAAGGCACTGGGTATATGGTATCCCCAA |
| | | ACAAGAGACATAATCCCGGTCCTTAGGTAGTGCTAGTGTGGTCTGTAATATCT |
| | | TACTAAGGCCTTTGGTATACGACCCAGAGATAACACGATGCGTATTTTAGTTT |
| | | TGCAAAGAAGGGGTTTGGTCTCTGTGCCAGCTCTATAATTGTTTTGCTACGAT |
| | | TCCACTGAAACTCTTCGATCAAGCTACTTTATGTAAATCACTTCATTGTTTTAA |
| | | AGGAATAAACTTGATTATATTGTTTTTTTATTTGGCATAACTGTGATTCTTTTA |
| | | GGACAATTACTGTACACATTAAGGTGTATGTCAGATATTCATATTGACCCAAA |
| | | TGTGTAATATTCCAGTTTTCTCTGCATAAGTAATTAAAATATACTTAAAAATTA |
| | | ATAGTTTTATCTGGGTACAAATAAACAGGTGCCTGAACTAGTTCACAGACAAG |
| | | GAAACTTCTATGTAAAAATCACTATGATTTCTGAATTGCTATGTGAAACTACA |
| | | GATCTTTGGAACACTGTTTAGGTAGGGTGTTAAGACTTACACAGTACCTCGTT |
| | | TCTACACAGAGAAAGAAATGGCCATACTTCAGGAACTGCAGTGCTTATGAGG |
| | | GGATATTTAGGCCTCTTGAATTTTTGATGTAGATGGGCATTTTTTTAAGGTAGT |
| | | GGTTAATTACCTTTATGTGAACTTTGAATGGTTTAACAAAAGATTTGTTTTTGT |
| | | AGAGATTTTAAAGGGGGAGAATTCTAGAAATAAATGTTACCTAATTATTACA |
| | | GCCTTAAAGACAAAAATCCTTGTTGAAGTTTTTTTAAAAAAAGCTAAATTACA |
| | | TAGACTTAGGCATTAACATGTTTGTGGAAGAATATAGCAGACGTATATTGTAT |
| | | CATTTGAGTGAATGTTCCCAAGTAGGCATTCTAGGCTCTATTTAACTGAGTCA |
| | | CACTGCATAGGAATTTAGAACCTAACTTTTATAGGTTATCAAAACTGTTGTCA |
| | | CCATTGCACAATTTTGTCCTAATATATACATAGAAACTTTGTGGGGCATGTTA |
| | | AGTTACAGTTTGCACAAGTTCATCTCATTTGTATTCCATTGATTTTTTTTTTCTT |
| | | CTAAACATTTTTTCTTCAAACAGTATATAACTTTTTTTAGGGGATTTTTTTTTA |
| | | GACAGCAAAAACTATCTGAAGATTTCCATTTGTCAAAAAGTAATGATTTCTTG |
| | | ATAATTGTGTAGTAATGTTTTTTAGAACCCAGCAGTTACCTTAAAGCTGAATT |
| | | TATATTTAGTAACTTCTGTGTTAATACTGGATAGCATGAATTCTGCATTGAGA |
| | | AACTGAATAGCTGTCATAAAATGAAACTTTCTTTCTAAAGAAAGATACTCACA |
| | | TG |
| SEQ ID | G12D | CTAGGCGGCGGCCGCGGCGGCGGAGGCAGCAGCGGCGGCGGCAGTGGCGGC |
| NO: 22 | | GGCGAAGGTGGCGGCGGCTCGGCCAGTACTCCCGGCCCCCGCCATTTCGGAC |
| | | TGGGAGCGAGCGCGGCGCAGGCACTGAAGGCGGCGGGGGGCCAGAGGCTC |
| | | AGCGGCTCCCAGGTGCGGGAGAGAGGCCTGCTGAAAATGACTGAATATAAAC |
| | | TTGTGGTAGTTGGAGCTGATGGCGTAGGCAAGAGTGCCTTGACGATACAGCT |
| | | AATTCAGAATCATTTTGTGGACGAATATGATCCAACAATAGAGGATTCCTACA |
| | | GGAAGCAAGTAGTAATTGATGGAGAAACCTGTCTCTTGGATATTCTCGACACA |
| | | GCAGGTCAAGAGGAGTACAGTGCAATGAGGGACCAGTACATGAGGACTGGG |
| | | GAGGGCTTTCTTTGTGTATTTGCCATAAATAATACTAAATCATTTGAAGATATT |
| | | CACCATTATAGAGAACAAATTAAAAGAGTTAAGGACTCTGAAGATGTACCTA |
| | | TGGTCCTAGTAGGAAATAAATGTGATTTGCCTTCTAGAACAGTAGACACAAA |
| | | ACAGGCTCAGGACTTAGCAAGAAGTTATGGAATTCCTTTTATTGAAACATCAG |
| | | CAAAGACAAGACAGAGAGTGGAGGATGCTTTTTATACATTGGTGAGAGAGAT |
| | | CCGACAATACAGATTGAAAAAAATCAGCAAAGAAGAAAAGACTCCTGGCTGT |
| | | GTGAAAATTAAAAAATGCATTATAATGTAATCTGGGTGTTGATGATGCCTTCT |
| | | ATACATTAGTTCGAGAAATTCGAAAACATAAAGAAAAGATGAGCAAAGATGG |
| | | TAAAAAGAAGAAAAAGAAGTCAAAGACAAAGTGTGTAATTATGTAAATACA |
| | | ATTTGTACTTTTTTCTTAAGGCATACTAGTACAAGTGGTAATTTTTGTACATTA |
| | | CACTAAATTATTAGCATTTGTTTTAGCATTACCTAATTTTTTTCCTGCTCCATG |
| | | CAGACTGTTAGCTTTTACCTTAAATGCTTATTTTAAAATGACAGTGGAAGTTTT |
| | | TTTTTCCTCTAAGTGCCAGTATTCCCAGAGTTTTGGTTTTTGAACTAGCAATGC |
| | | CTGTGAAAAAGAAACTGAATACCTAAGATTTCTGTCTTGGGGCTTTTGGTGCA |
| | | TGCAGTTGATTACTTCTTATTTTTCTTACCAATTGTGAATGTTGGTGTGAAACA |
| | | AATTAATGAAGCTTTTGAATCATCCCTATTCTGTGTTTTATCTAGTCACATAAA |
| | | TGGATTAATTACTAATTTCAGTTGAGACCTTCTAATTGGTTTTTACTGAAACAT |
| | | TGAGGGAACACAAATTTATGGGCTTCCTGATGATGATTCTTCTAGGCATCATG |
| | | TCCTATAGTTTGTCATCCCTGATGAATGTAAAGTTACACTGTTCACAAAGGTTT |
| | | TGTCTCCTTTCCACTGCTATTAGTCATGGTCACTCTCCCCAAAATATTATATTT |
| | | TTTCTATAAAAAGAAAAAAATGGAAAAAAATTACAAGGCAATGGAAACTATT |
| | | ATAAGGCCATTTCCTTTTCACATTAGATAAATTACTATAAAGACTCCTAATAG |
| | | CTTTTCCTGTTAAGGCAGACCCAGTATGAAATGGGGATTATTATAGCAACCAT |
| | | TTTGGGGCTATATTTACATGCTACTAAATTTTTATAATAATTGAAAAGATTTTA |
| | | ACAAGTATAAAAAATTCTCATAGGAATTAAATGTAGTCTCCCTGTGTCAGACT |
| | | GCTCTTTCATAGTATAACTTTAAATCTTTTCTTCAACTTGAGTCTTTGAAGATA |
| | | GTTTTAATTCTGCTTGTGACATTAAAAGATTATTTGGGCCAGTTATAGCTTATT |
| | | AGGTGTTGAAGAGACCAAGGTTGCAAGGCCAGGCCCTGTGTGAACCTTTGAG |
| | | CTTTCATAGAGAGTTTCACAGCATGGACTGTGTCCCCACGGTCATCCAGTGTT |
| | | GTCATGCATTGGTTAGTCAAAATGGGGAGGGACTAGGGCAGTTTGGATAGCT |
| | | CAACAAGATACAATCTCACTCTGTGGTGGTCCTGCTGACAAATCAAGAGCATT |
| | | GCTTTTGTTTCTTAAGAAAACAAACTCTTTTTTAAAAATTACTTTTAAATATTA |
| | | ACTCAAAAGTTGAGATTTTGGGGTGGTGGTGTGCCAAGACATTAATTTTTTTT |
| | | TTAAACAATGAAGTGAAAAAGTTTTACAATCTCTAGGTTTGGCTAGTTCTCTT |
| | | AACACTGGTTAAATTAACATTGCATAAACACTTTTCAAGTCTGATCCATATTT |
| | | AATAATGCTTTAAAATAAAAATAAAAACAATCCTTTTGATAAATTTAAAATGT |
| | | TACTTATTTTAAAATAAATGAAGTGAGATGGCATGGTGAGGTGAAAGTATCA |
| | | CTGGACTAGGAAGAAGGTGACTTAGGTTCTAGATAGGTGTCTTTTAGGACTCT |
| | | GATTTTGAGGACATCACTTACTATCCATTTCTTCATGTTAAAAGAAGTCATCTC |
| | | AAACTCTTAGTTTTTTTTTTTTACAACTATGTAATTTATATTCCATTTACATAAG |
| | | GATACACTTATTTGTCAAGCTCAGCACAATCTGTAAATTTTTAACCTATGTTAC |
| | | ACCATCTTCAGTGCCAGTCTTGGGCAAAATTGTGCAAGAGGTGAAGTTTATAT |
| | | TTGAATATCCATTCTCGTTTTAGGACTCTTCTTCCATATTAGTGTCATCTTGCCT |
| | | CCCTACCTTCCACATGCCCCATGACTTGATGCAGTTTTAATACTTGTAATTCCC |
| | | CTAACCATAAGATTTACTGCTGCTGTGGATATCTCCATGAAGTTTTCCCACTG |
| | | AGTCACATCAGAAATGCCCTACATCTTATTTCCTCAGGGCTCAAGAGAATCTG |
| | | ACAGATACCATAAAGGGATTTGACCTAATCACTAATTTTCAGGTGGTGGCTGA |
| | | TGCTTTGAACATCTCTTTGCTGCCCAATCCATTAGCGACAGTAGGATTTTTCAA |
| | | ACCTGGTATGAATAGACAGAACCCTATCCAGTGGAAGGAGAATTTAATAAAG |
| | | ATAGTGCTGAAAGAATTCCTTAGGTAATCTATAACTAGGACTACTCCTGGTAA |
| | | CAGTAATACATTCCATTGTTTTAGTAACCAGAAATCTTCATGCAATGAAAAAT |
| | | ACTTTAATTCATGAAGCTTACTTTTTTTTTTTGGTGTCAGAGTCTCGCTCTTGTC |
| | | ACCCAGGCTGGAATGCAGTGGCGCCATCTCAGCTCACTGCAACCTCCATCTCC |
| | | CAGGTTCAAGCGATTCTCGTGCCTCGGCCTCCTGAGTAGCTGGGATTACAGGC |
| | | GTGTGCCACTACACTCAACTAATTTTTGTATTTTTAGGAGAGACGGGGTTTCA |
| | | CCCTGTTGGCCAGGCTGGTCTCGAACTCCTGACCTCAAGTGATTCACCCACCT |
| | | TGGCCTCATAAACCTGTTTTGCAGAACTCATTTATTCAGCAAATATTTATTGAG |
| | | TGCCTACCAGATGCCAGTCACCACACAAGGCACTGGGTATATGGTATCCCCAA |
| | | ACAAGAGACATAATCCCGGTCCTTAGGTAGTGCTAGTGTGGTCTGTAATATCT |
| | | TACTAAGGCCTTTGGTATACGACCCAGAGATAACACGATGCGTATTTTAGTTT |
| | | TGCAAAGAAGGGGTTTGGTCTCTGTGCCAGCTCTATAATTGTTTTGCTACGAT |
| | | TCCACTGAAACTCTTCGATCAAGCTACTTTATGTAAATCACTTCATTGTTTTAA |
| | | AGGAATAAACTTGATTATATTGTTTTTTTATTTGGCATAACTGTGATTCTTTTA |
| | | GGACAATTACTGTACACATTAAGGTGTATGTCAGATATTCATATTGACCCAAA |
| | | TGTGTAATATTCCAGTTTTCTCTGCATAAGTAATTAAAATATACTTAAAAATTA |
| | | ATAGTTTTATCTGGGTACAAATAAACAGGTGCCTGAACTAGTTCACAGACAAG |
| | | GAAACTTCTATGTAAAAATCACTATGATTTCTGAATTGCTATGTGAAACTACA |
| | | GATCTTTGGAACACTGTTTAGGTAGGGTGTTAAGACTTACACAGTACCTCGTT |
| | | TCTACACAGAGAAAGAAATGGCCATACTTCAGGAACTGCAGTGCTTATGAGG |
| | | GGATATTTAGGCCTCTTGAATTTTTGATGTAGATGGGCATTTTTTTAAGGTAGT |
| | | GGTTAATTACCTTTATGTGAACTTTGAATGGTTTAACAAAAGATTTGTTTTTGT |
| | | AGAGATTTTAAAGGGGGAGAATTCTAGAAATAAATGTTACCTAATTATTACA |
| | | GCCTTAAAGACAAAAATCCTTGTTGAAGTTTTTTTAAAAAAAGCTAAATTACA |
| | | TAGACTTAGGCATTAACATGTTTGTGGAAGAATATAGCAGACGTATATTGTAT |
| | | CATTTGAGTGAATGTTCCCAAGTAGGCATTCTAGGCTCTATTTAACTGAGTCA |
| | | CACTGCATAGGAATTTAGAACCTAACTTTTATAGGTTATCAAAACTGTTGTCA |
| | | CCATTGCACAATTTTGTCCTAATATATACATAGAAACTTTGTGGGGCATGTTA |
| | | AGTTACAGTTTGCACAAGTTCATCTCATTTGTATTCCATTGATTTTTTTTTTCTT |
| | | CTAAACATTTTTTCTTCAAACAGTATATAACTTTTTTTAGGGGATTTTTTTTTA |
| | | GACAGCAAAAACTATCTGAAGATTTCCATTTGTCAAAAAGTAATGATTTCTTG |
| | | ATAATTGTGTAGTAATGTTTTTTAGAACCCAGCAGTTACCTTAAAGCTGAATT |
| | | TATATTTAGTAACTTCTGTGTTAATACTGGATAGCATGAATTCTGCATTGAGA |
| | | AACTGAATAGCTGTCATAAAATGAAACTTTCTTTCTAAAGAAAGATACTCACA |
| | | TG |
| SEQ ID | G12V | CTAGGCGGCGGCCGCGGCGGCGGAGGCAGCAGCGGCGGCGGCAGTGGCGGC |
| NO: 23 | | GGCGAAGGTGGCGGCGGCTCGGCCAGTACTCCCGGCCCCCGCCATTTCGGAC |
| | | TGGGAGCGAGCGCGGCGCAGGCACTGAAGGCGGCGGCGGGGCCAGAGGCTC |
| | | AGCGGCTCCCAGGTGCGGGAGAGAGGCCTGCTGAAAATGACTGAATATAAAC |
| | | TTGTGGTAGTTGGAGCTGTTGGCGTAGGCAAGAGTGCCTTGACGATACAGCTA |
| | | ATTCAGAATCATTTTGTGGACGAATATGATCCAACAATAGAGGATTCCTACAG |
| | | GAAGCAAGTAGTAATTGATGGAGAAACCTGTCTCTTGGATATTCTCGACACAG |
| | | CAGGTCAAGAGGAGTACAGTGCAATGAGGGACCAGTACATGAGGACTGGGG |
| | | AGGGCTTTCTTTGTGTATTTGCCATAAATAATACTAAATCATTTGAAGATATTC |
| | | ACCATTATAGAGAACAAATTAAAAGAGTTAAGGACTCTGAAGATGTACCTAT |
| | | GGTCCTAGTAGGAAATAAATGTGATTTGCCTTCTAGAACAGTAGACACAAAA |
| | | CAGGCTCAGGACTTAGCAAGAAGTTATGGAATTCCTTTTATTGAAACATCAGC |
| | | AAAGACAAGACAGAGAGTGGAGGATGCTTTTTATACATTGGTGAGAGAGATC |
| | | CGACAATACAGATTGAAAAAAATCAGCAAAGAAGAAAAGACTCCTGGCTGTG |
| | | TGAAAATTAAAAAATGCATTATAATGTAATCTGGGTGTTGATGATGCCTTCTA |
| | | TACATTAGTTCGAGAAATTCGAAAACATAAAGAAAAGATGAGCAAAGATGGT |
| | | AAAAAGAAGAAAAAGAAGTCAAAGACAAAGTGTGTAATTATGTAAATACAA |
| | | TTTGTACTTTTTTCTTAAGGCATACTAGTACAAGTGGTAATTTTTGTACATTAC |
| | | ACTAAATTATTAGCATTTGTTTTAGCATTACCTAATTTTTTTCCTGCTCCATGC |
| | | AGACTGTTAGCTTTTACCTTAAATGCTTATTTTAAAATGACAGTGGAAGTTTTT |
| | | TTTTCCTCTAAGTGCCAGTATTCCCAGAGTTTTGGTTTTTGAACTAGCAATGCC |
| | | TGTGAAAAAGAAACTGAATACCTAAGATTTCTGTCTTGGGGCTTTTGGTGCAT |
| | | GCAGTTGATTACTTCTTATTTTTCTTACCAATTGTGAATGTTGGTGTGAAACAA |
| | | ATTAATGAAGCTTTTGAATCATCCCTATTCTGTGTTTTATCTAGTCACATAAAT |
| | | GGATTAATTACTAATTTCAGTTGAGACCTTCTAATTGGTTTTTACTGAAACATT |
| | | GAGGGAACACAAATTTATGGGCTTCCTGATGATGATTCTTCTAGGCATCATGT |
| | | CCTATAGTTTGTCATCCCTGATGAATGTAAAGTTACACTGTTCACAAAGGTTTT |
| | | GTCTCCTTTCCACTGCTATTAGTCATGGTCACTCTCCCCAAAATATTATATTTT |
| | | TTCTATAAAAAGAAAAAAATGGAAAAAAATTACAAGGCAATGGAAACTATTA |
| | | TAAGGCCATTTCCTTTTCACATTAGATAAATTACTATAAAGACTCCTAATAGC |
| | | TTTTCCTGTTAAGGCAGACCCAGTATGAAATGGGGATTATTATAGCAACCATT |
| | | TTGGGGCTATATTTACATGCTACTAAATTTTTATAATAATTGAAAAGATTTTAA |
| | | CAAGTATAAAAAATTCTCATAGGAATTAAATGTAGTCTCCCTGTGTCAGACTG |
| | | CTCTTTCATAGTATAACTTTAAATCTTTTCTTCAACTTGAGTCTTTGAAGATAG |
| | | TTTTAATTCTGCTTGTGACATTAAAAGATTATTTGGGCCAGTTATAGCTTATTA |
| | | GGTGTTGAAGAGACCAAGGTTGCAAGGCCAGGCCCTGTGTGAACCTTTGAGC |
| | | TTTCATAGAGAGTTTCACAGCATGGACTGTGTCCCCACGGTCATCCAGTGTTG |
| | | TCATGCATTGGTTAGTCAAAATGGGGAGGGACTAGGGCAGTTTGGATAGCTC |
| | | AACAAGATACAATCTCACTCTGTGGTGGTCCTGCTGACAAATCAAGAGCATTG |
| | | CTTTTGTTTCTTAAGAAAACAAACTCTTTTTTAAAAATTACTTTTAAATATTAA |
| | | CTCAAAAGTTGAGATTTTGGGGTGGTGGTGTGCCAAGACATTAATTTTTTTTTT |
| | | AAACAATGAAGTGAAAAAGTTTTACAATCTCTAGGTTTGGCTAGTTCTCTTAA |
| | | CACTGGTTAAATTAACATTGCATAAACACTTTTCAAGTCTGATCCATATTTAAT |
| | | AATGCTTTAAAATAAAAATAAAAACAATCCTTTTGATAAATTTAAAATGTTAC |
| | | TTATTTTAAAATAAATGAAGTGAGATGGCATGGTGAGGTGAAAGTATCACTG |
| | | GACTAGGAAGAAGGTGACTTAGGTTCTAGATAGGTGTCTTTTAGGACTCTGAT |
| | | TTTGAGGACATCACTTACTATCCATTTCTTCATGTTAAAAGAAGTCATCTCAA |
| | | ACTCTTAGTTTTTTTTTTTTACAACTATGTAATTTATATTCCATTTACATAAGGA |
| | | TACACTTATTTGTCAAGCTCAGCACAATCTGTAAATTTTTAACCTATGTTACAC |
| | | CATCTTCAGTGCCAGTCTTGGGCAAAATTGTGCAAGAGGTGAAGTTTATATTT |
| | | GAATATCCATTCTCGTTTTAGGACTCTTCTTCCATATTAGTGTCATCTTGCCTC |
| | | CCTACCTTCCACATGCCCCATGACTTGATGCAGTTTTAATACTTGTAATTCCCC |
| | | TAACCATAAGATTTACTGCTGCTGTGGATATCTCCATGAAGTTTTCCCACTGA |
| | | GTCACATCAGAAATGCCCTACATCTTATTTCCTCAGGGCTCAAGAGAATCTGA |
| | | CAGATACCATAAAGGGATTTGACCTAATCACTAATTTTCAGGTGGTGGCTGAT |
| | | GCTTTGAACATCTCTTTGCTGCCCAATCCATTAGCGACAGTAGGATTTTTCAA |
| | | ACCTGGTATGAATAGACAGAACCCTATCCAGTGGAAGGAGAATTTAATAAAG |
| | | ATAGTGCTGAAAGAATTCCTTAGGTAATCTATAACTAGGACTACTCCTGGTAA |
| | | CAGTAATACATTCCATTGTTTTAGTAACCAGAAATCTTCATGCAATGAAAAAT |
| | | ACTTTAATTCATGAAGCTTACTTTTTTTTTTTGGTGTCAGAGTCTCGCTCTTGTC |
| | | ACCCAGGCTGGAATGCAGTGGCGCCATCTCAGCTCACTGCAACCTCCATCTCC |
| | | CAGGTTCAAGCGATTCTCGTGCCTCGGCCTCCTGAGTAGCTGGGATTACAGGC |
| | | GTGTGCCACTACACTCAACTAATTTTTGTATTTTTAGGAGAGACGGGGTTTCA |
| | | CCCTGTTGGCCAGGCTGGTCTCGAACTCCTGACCTCAAGTGATTCACCCACCT |
| | | TGGCCTCATAAACCTGTTTTGCAGAACTCATTTATTCAGCAAATATTTATTGAG |
| | | TGCCTACCAGATGCCAGTCACCACACAAGGCACTGGGTATATGGTATCCCCAA |
| | | ACAAGAGACATAATCCCGGTCCTTAGGTAGTGCTAGTGTGGTCTGTAATATCT |
| | | TACTAAGGCCTTTGGTATACGACCCAGAGATAACACGATGCGTATTTTAGTTT |
| | | TGCAAAGAAGGGGTTTGGTCTCTGTGCCAGCTCTATAATTGTTTTGCTACGAT |
| | | TCCACTGAAACTCTTCGATCAAGCTACTTTATGTAAATCACTTCATTGTTTTAA |
| | | AGGAATAAACTTGATTATATTGTTTTTTTATTTGGCATAACTGTGATTCTTTTA |
| | | GGACAATTACTGTACACATTAAGGTGTATGTCAGATATTCATATTGACCCAAA |
| | | TGTGTAATATTCCAGTTTTCTCTGCATAAGTAATTAAAATATACTTAAAAATTA |
| | | ATAGTTTTATCTGGGTACAAATAAACAGGTGCCTGAACTAGTTCACAGACAAG |
| | | GAAACTTCTATGTAAAAATCACTATGATTTCTGAATTGCTATGTGAAACTACA |
| | | GATCTTTGGAACACTGTTTAGGTAGGGTGTTAAGACTTACACAGTACCTCGTT |
| | | TCTACACAGAGAAAGAAATGGCCATACTTCAGGAACTGCAGTGCTTATGAGG |
| | | GGATATTTAGGCCTCTTGAATTTTTGATGTAGATGGGCATTTTTTTAAGGTAGT |
| | | GGTTAATTACCTTTATGTGAACTTTGAATGGTTTAACAAAAGATTTGTTTTTGT |
| | | AGAGATTTTAAAGGGGGAGAATTCTAGAAATAAATGTTACCTAATTATTACA |
| | | GCCTTAAAGACAAAAATCCTTGTTGAAGTTTTTTTAAAAAAAGCTAAATTACA |
| | | TAGACTTAGGCATTAACATGTTTGTGGAAGAATATAGCAGACGTATATTGTAT |
| | | CATTTGAGTGAATGTTCCCAAGTAGGCATTCTAGGCTCTATTTAACTGAGTCA |
| | | CACTGCATAGGAATTTAGAACCTAACTTTTATAGGTTATCAAAACTGTTGTCA |
| | | CCATTGCACAATTTTGTCCTAATATATACATAGAAACTTTGTGGGGCATGTTA |
| | | AGTTACAGTTTGCACAAGTTCATCTCATTTGTATTCCATTGATTTTTTTTTTCTT |
| | | CTAAACATTTTTTCTTCAAACAGTATATAACTTTTTTTAGGGGATTTTTTTTTA |
| | | GACAGCAAAAACTATCTGAAGATTTCCATTTGTCAAAAAGTAATGATTTCTTG |
| | | ATAATTGTGTAGTAATGTTTTTTAGAACCCAGCAGTTACCTTAAAGCTGAATT |
| | | TATATTTAGTAACTTCTGTGTTAATACTGGATAGCATGAATTCTGCATTGAGA |
| | | AACTGAATAGCTGTCATAAAATGAAACTTTCTTTCTAAAGAAAGATACTCACA |
| | | TG |

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

What is claimed is:

1. A compound comprising an antisense oligonucleotide that inhibits the expression of a KRAS mRNA, wherein the antisense oligonucleotide comprises 10 to 30 linked nucleotides and has a sequence that is complementary to a KRAS mRNA, and wherein the oligonucleotide has at least 8 contiguous nucleotides of any one of SEQ ID NOS: 1-10 and 14-21.

2. The compound of claim 2, wherein the oligonucleotide is at least 12 nucleotides in length.

3. The compound of claim 3, wherein the oligonucleotide is at least 14 nucleotides in length.

4. The compound of claim 2, wherein the oligonucleotide is from 10 to 24 nucleotides in length, or 10 to 16 nucleotides in length, or 12 to 16 nucleotides in length.

5. The compound of claim 5, wherein the oligonucleotide is 12, 13, 14, 15, or 16 nucleotides in length.

6. The compound of claim 6, wherein the oligonucleotide is 14 nucleotides in length.

7. The compound of any one of claims 1 to 6, wherein the oligonucleotide comprises at least 12 contiguous nucleobases of any one of SEQ ID NOs: 1-10 and 14-21.

8. The compound of claim 7, wherein the oligonucleotide comprises or consists of a nucleobase sequence of any one of SEQ ID NOs: 1-10 and 14-21.

9. The compound of claim 8, wherein the oligonucleotide has a nucleobase sequence selected from SEQ ID NOs: 2, 3, 4, 5, 6, 7, 8, 9, and 10.

10. The compound of any one of claims 1 to 9, wherein the antisense oligonucleotide has a stretch of at least 6 DNA nucleotides sufficient to recruit RNaseH.

11. The compound of claim 10, wherein one or more DNA nucleotides comprise a 2′ chemical modification independently selected from 2′-Fluoro, 2′-Methyl, and 2′-Ethyl.

12. The compound of claim 10, wherein DNA nucleotides do not comprise a 2′ chemical modification.

13. The compound of any one of claims 10 to 12, wherein the antisense oligonucleotide is a gapmer having a 5′ and a 3′ segment, each of the 5′ and 3′ segments being from 2 to 6 nucleotides or from 2 to 4 nucleotides, and where the 5′ and 3′ segments do not contain DNA nucleotides.

14. The compound of claim 13, wherein the 5′ and 3′ segments are each independently selected from 2 or 3 nucleotides in length, and the 5′ and 3′ segments flank an internal sequence of 8 DNA nucleotides.

15. The compound of claim 14, wherein one or more nucleotides of the 5′ segment and the 3′ segment comprise 2′-O substituents, optionally where all of the nucleotides of the 5′ segment and the 3′ segment comprise 2′-O substituents.

16. The compound of claim 15, wherein the 2′-O substituents are independently selected from 2′-O methyl, 2′-O ethyl, 2′-O methoxyethyl (MOE), and a bridged nucleotide having a 2′ to 4′ bridge.

17. The compound of claim 16, wherein the bridged nucleotide has a methylene bridge (LNA) or a constrained ethyl bridge (cEt).

18. The compound of any one of claims 1 to 17, wherein the antisense oligonucleotide has a modified backbone.

19. The compound of claim 18, wherein the antisense oligonucleotide comprises one or more phosphorothioate or phosphorodithioate nucleotides.

20. The compound of claim 19, wherein the oligonucleotide is fully phosphorothioate or phosphorodithioate linked.

21. The compound of claim 20, wherein the oligonucleotide is fully phosphorothioate linked.

22. The compound of any one of claims 1 to 21, wherein cytosine nucleobases in the antisense oligonucleotide are modified cytosine, which is optionally 5-methyl cytosine or 5-hydroxymethyl cytosine.

23. The compound of claim 1, wherein the antisense oligonucleotide has a structure shown in one or more of Tables 1, 2, 3, 6, 7, 8, 9, 10, and 11.

24. The compound of any one of claims 1 to 23, further comprising a cell targeting or penetrating moiety.

25. The compound of claim 24, wherein the cell targeting or penetrating moiety is conjugated directly or indirectly at the 3′ end of the oligonucleotide, optionally though a linker.

26. The compound of claim 24 or claim 25, wherein the moiety comprises a sterol conjugate or fatty acid conjugate, which is optionally cholesteryl, palmitoyl, or stearyl conjugate.

27. The compound of claim 24, wherein the compound further comprises a cell targeting aptamer.

28. The compound of any one of claims 1 to 27, wherein the compound does not comprise any encapsulation or transfection reagent.

29. The compound of any one of claims 1 to 27, wherein the antisense oligonucleotide is encapsulated in a particle.

30. The compound of claim 29, wherein the particle is a liposome, polymeric nanoparticle, or lipid nanoparticle.

31. The compound of any one of claims 1 to 30, wherein the compound is formulated for parenteral administration.

32. A pharmaceutical composition comprising a compound of any one of claims 1 to 31, and a pharmaceutically acceptable carrier or vehicle.

33. A method for treating a subject having a condition associated with aberrant expression of KRAS or associated with mutated KRAS, comprising administering an effective amount of the compound of any one of claims 1 to 32, or the pharmaceutical composition of claim 32, to the subject.

34. The method of claim 33, wherein the subject has a malignancy associated with an abnormality of KRAS-mediated signaling pathway.

35. The method of claim 34, wherein the malignancy is associated with KRAS or a mutated KRAS.

36. The method of claim 35, wherein the mutated KRAS mRNA encodes a mutated KRAS protein comprising a G12C mutation.

37. The method of claim 35, wherein the mutated KRAS mRNA encodes a mutated KRAS protein comprising a G12D mutation or a G12V mutation.

38. The method of any one of claims 34 to 37, wherein the malignancy associated with an abnormality of KRAS-mediated signaling pathway is non-metastatic.

39. The method of any one of claims 34 to 37, wherein the malignancy associated with an abnormality of KRAS-mediated signaling pathway is metastatic.

40. The method of any one of claims 34 to 39, wherein the malignancy is a carcinoma.

41. The method of claim 40, wherein the malignancy is breast cancer, cervical cancer, pancreatic cancer, squamous cell carcinoma, head and neck cancer, thyroid cancer, gastric cancer, colon cancer, or liver cancer.

42. The method of claim 41, wherein the malignancy is pancreatic cancer.

43. The method of claim 40, wherein the malignancy is cervical squamous cell carcinoma, endocervical adenocarcinoma, cholangiocarcinoma, esophageal carcinoma, colon adenocarcinoma, oral squamous cell carcinoma, head and neck squamous cell carcinoma, lung adenocarcinoma, lung squamous cell carcinoma, non-small cell lung cancer, liver hepatocellular carcinoma, lung squamous cell carcinoma, rectum adenocarcinoma, stomach adenocarcinoma, thyroid carcinoma, or pancreatic adenocarcinoma.

44. The method of any one of claims 33 to 43, wherein the compound or composition is administered parenterally.


SEQUENCE LISTING (23 sequences)

SEQ ID NO: 1 - DNA, 16 bp, synthetic construct
  source 1..16: mol_type = other DNA
cctcattgcactgtac

SEQ ID NO: 2 - DNA, 14 bp, synthetic construct
  source 1..14: mol_type = other DNA
tacgccaacagctc

SEQ ID NO: 3 - DNA, 14 bp, synthetic construct
  source 1..14: mol_type = other DNA
ctacgccaacagct

SEQ ID NO: 4 - DNA, 12 bp, synthetic construct
  source 1..12: mol_type = other DNA
agccaacagctc

SEQ ID NO: 5 - DNA, 13 bp, synthetic construct
  source 1..13: mol_type = other DNA
acgccaacagctc

SEQ ID NO: 6 - DNA, 13 bp, synthetic construct
  source 1..13: mol_type = other DNA
agccatcagctca

SEQ ID NO: 7 - DNA, 14 bp, synthetic construct
  source 1..14: mol_type = other DNA
tacgccacaagctc

SEQ ID NO: 8 - DNA, 13 bp, synthetic construct
  source 1..13: mol_type = other DNA
acgccacaagctc

SEQ ID NO: 9 - DNA, 14 bp, synthetic construct
  source 1..14: mol_type = other DNA
acgccacaagctcc

SEQ ID NO: 10 - DNA, 15 bp, synthetic construct
  source 1..15: mol_type = other DNA
acgccatcagctcca

SEQ ID NO: 11 - DNA, 4835 bp, synthetic construct
  source 1..4835: mol_type = other DNA
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatg

SEQ ID NO: 12 - DNA, 4835 bp, synthetic construct
  source 1..4835: mol_type = other DNA
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctgctggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatg

SEQ ID NO: 13 - DNA, 4835 bp, synthetic construct
  source 1..4835: mol_type = other DNA
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagcttgtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatg

SEQ ID NO: 14 - DNA, 14 bp, synthetic construct
  source 1..14: mol_type = other DNA
ctacgccacaagct

SEQ ID NO: 15 - DNA, 15 bp, synthetic construct
  source 1..15: mol_type = other DNA
ctacgccacaagctc

SEQ ID NO: 16 - DNA, 16 bp, synthetic construct
  source 1..16: mol_type = other DNA
tacgccacaagctcca

SEQ ID NO: 17 - DNA, 15 bp, synthetic construct
  source 1..15: mol_type = other DNA
acgccacaagctcca

SEQ ID NO: 18 - DNA, 14 bp, synthetic construct
  source 1..14: mol_type = other DNA
cgccacaagctcca

SEQ ID NO: 19 - DNA, 13 bp, synthetic construct
  source 1..13: mol_type = other DNA
cgccacaagctcc

SEQ ID NO: 20 - DNA, 14 bp, synthetic construct
  source 1..14: mol_type = other DNA
acgtctatacacca

SEQ ID NO: 21 - DNA, 16 bp, synthetic construct
  source 1..16: mol_type = other DNA
cacgtctatacaccac

SEQ ID NO: 22 - DNA, 4835 bp, synthetic construct
  source 1..4835: mol_type = other DNA
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctgatggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatg

SEQ ID NO: 23 - DNA, 4835 bp, synthetic construct
  source 1..4835: mol_type = other DNA
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctgttggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatg